(19) United States
(12) Patent Application Publication
(10) Pub. No.: US 2026/0229329 A1
(43) Pub. Date: Aug. 6, 2026

(54) Artificial Intelligence/Machine Learning for Enhanced Logging and Reporting of Events for Individuals Under Care, Including Reviewing and Escalating Documentation, and Systems and Methods Therefor

(71) Applicant: Therap Services, LLC, Torrington, CT (US)

(72) Inventors: Justin Mark Brockie, Wolcott, CT (US); David Lawrence Turock, Fort Lauderdale, FL (US); Md Rumman Bin Ashraf, Dhaka (BD); Fahim Faisal, Dhaka (BD); Md. Nazrul Islam, Dhaka (BD); James Michael Kelly, Morris, CT (US); Touhidur Rahaman Khan, Dhaka (BD); Jason Douglas Laws, Athens, GA (US); Richard Allen Robbins, Lenox, MA (US); Md Tahmid Shakoor, Dhaka (BD); William Edward Sepesi, San Francisco, CA (US); James Joseph Brockman, Millstone Township, NJ (US)

(73) Assignee: Therap Services, LLC, Torrington, CT (US)

(21) Appl. No.: 19/278,858

(22) Filed: Jul. 24, 2025

Related U.S. Application Data
(60) Provisional application No. 63/751,874, filed on Jan. 31, 2025.

(51) Int. Cl. G16H 15/00; G16H 10/60

(52) U.S. Cl. CPC G16H 15/00; G16H 10/60

(57) ABSTRACT

Artificial Intelligence/machine learning is used to improve HIPAA-compliant computer systems and methods for linking electronic GER and T-Log records relating to individuals under care by a caregiver. T-log records are compared to GER subject matters to determine whether an accuracy score exceeds a threshold. If so, the T-Log record is compared to GER events to determine whether an accuracy score exceeds a threshold. If so, the T-Log record is compared to GER subtypes to determine whether an accuracy score exceeds a threshold. If so, the T-Log record is compared to existing GER records to determine whether an accuracy score exceeds a threshold. If so, the T-Log and GER records are electronically linked and an alert is provided to the caregiver. One or more of these comparisons is performed at least in part by categorizing said T-Log record by using said one or more large language models.

### CROSS-REFERENCE TO RELATED APPLICATIONS

This application claims, and is entitled to claim, a right of priority to U.S. Provisional Patent Application Ser. No. 63/751,874 filed Jan. 31, 2025, and is entitled to the benefit of the filing date thereof.

This application incorporates the entirety of the following U.S. Patent and Patent Applications:
U.S. Pat. No. 12,217,316 filed as U.S. patent application Ser. No. 17/827,521 on May 27, 2022 (“the '316 Patent”);
U.S. Pat. No. 11,915,806, filed as U.S. patent application Ser. No. 17/941,329 on Sep. 9, 2022 (“the '806 Patent”);
U.S. Pat. No. 11,475,983 filed as U.S. patent application Ser. No. 16/695,591, on Nov. 25, 2019 (“the '983 Patent”);
U.S. Pat. No. 8,281,370 filed as U.S. patent application Ser. No. 11/604,577, on Nov. 27, 2006 (“the '370 Patent”); and
U.S. patent application Ser. No. 19/222,011 filed on May 29, 2025 (“the '011 Application”).

The '316 Patent is a continuation-in-part of U.S. Pat. No. 11,449,954 filed as U.S. patent application Ser. No. 16/750,388 on Jan. 23, 2020, which is a continuation-in-part of U.S. Pat. No. 10,586,290 filed as U.S. patent application Ser. No. 15/197,120 on Jun. 29, 2016, which is a continuation-in-part of U.S. patent application Ser. No. 13/675,440 (“the '440 Application”) filed Nov. 13, 2012. The '316 Patent is also a continuation-in-part of U.S. Pat. No. Ser. No. 11/410,759 filed as U.S. patent application Ser. No. 16/811,429 on Mar. 3, 2020, which is a continuation of U.S. Pat. No. 10,622,103 filed as U.S. patent application Ser. No. 15/636,826 on Jun. 6, 2017.

The '806 patent is a continuation of the '983 Patent, which claims priority to the '440 Application, which is a continuation-in-part of U.S. Pat. Nos. 8,615,790 and 8,813,054, both of which are divisions of the '370 Patent.

All description, drawings and teachings set forth in the '316, '806, '983, and '370 Patents and the '011 Application are expressly incorporated by reference herein.

### BACKGROUND OF THE INVENTION

When people think of Artificial Intelligence they are currently often thinking about products using generative AI to make suggestions such as music or movies. For example, if someone is using Spotify, according to the internet there are over 100 million songs with several billion playlists. Netflix or Hulu do similar things with TV shows and movies. They attempt to make suggestions based on personal interests using generative Ai and other tools. But what is interesting is that the threshold for getting a recommendation wrong is relatively low. If Netflix suggests 10 movies on a screen at one point and someone doesn't like one of the recommendations they can either not click on it or can hit next and not use a recommendation. But it is also the case that for any given song recommendation a person could have asked a friend or otherwise researched and produce the same song or movie or food or other recommendation. These AI engines are often replacing friends or guidebooks or other forms of recommendations. But keep in mind that, as an example, getting a Spotify recommendation wrong isn't harmful, but getting a recommendation for a person with intellectual and/or cognitive disabilities wrong could lead to a harmful-even fatal result. An example could be an individual with celiac disease getting fed a soft pretzel based on an AI-hallucinated care plan.

Providing services to people with cognitive disabilities creates special challenges. Provision of care must comply with federal and state regulations such as HIPAA and the Cares Act, as further described in the '983 Patent at Col. 2, Lines 64-3: 5, and the '316 Patent at Col. 3, Lines 21-35 and Col. 4, Lines 41-50 (collectively “HIPAA-type regulations”). There are many situations where people with cognitive disabilities or receiving long term support services need person-centered suggestions or documentation which can have significant impacts on their health, their goals and objectives, and also the finances of the agency supporting them if they don't have the proper recommendations or analysis. This information needs to be based on their exact situation and cannot be guesswork or an AI hallucination-or else there may be funding, compliance, medical or other complications. Because this involves person-specific personal health information (“PHI,” as may be defined in one or more HIPAA-type regulations), compliance with HIPAA-type regulations prevent generic systems from having access to this data and to make recommendations. At present much of this is being done in a limited manner by individual staff or supervisors or other workers trained to look at limited pieces of information. There is a need for an automated process which can look at data from staff, family, agencies, sensors, automated systems, external websites, APIs and more to general actual reports, suggestions, compliance material and more which cannot be done by hand.

There are many people receiving long term support and services either through government agencies, non-profit organizations, companies, community-based programs and through families and friends. This includes people with various cognitive and intellectual disabilities of many ages from birth to three, through school age, through adulthood and for the elderly. While different ages may require different levels and types of support, there are both commonalities of reportings and supports people need as well as person centered support based on each person's needs and specific information including medical, family, emotional, behavioral, and other conditions. Processes may also vary by government or funding jurisdiction.

In an optimal world all government funds are properly used, all people providing support for individuals receiving long term support and services would be properly trained and have the ability to instantly process the information, all compliance reports would be properly filled out, and data and information which would help improve positive outcomes and reduce problematic outcomes would be eliminated. However, that is not always how the supports for people receiving Home and Community Based Supports works. While most, although probably not all, people providing those services are well intentioned, they often cannot provide the proper services and required documentation in any sort of real-time manner. In many instances there are issues regarding types and quality of support. These problems can be seen on an individual staffing level or at an agency (non-profit or for-profit) providing these services often under contract with a government agency or other funding source. For example, most states have regulations and requirements for agencies to provide real time documentation for events such as death, abuse and neglect, medication errors, falls, and other events. One way this documentation can be fulfilled is through an online incident reporting system, such as those shown in Appendix 2. An agency, for example, could give access to a state or oversight agency to review this documentation in real time. However, if an incident occurs and there is not a report by the agency providing services to the oversight or state organization, there could be a problem both in the quality of service provided to the individual with disabilities and also there could be a problem for the agency which might receive a citation.

Most agencies try to comply with these regulations. For example, if there is a death, a fall, an injury, a hospital visit, the agency in almost all instances will not be trying to hide this event. It will have happened and it is in the agency's interest to report it and take action to support and care for an individual. But there are instances where the front-line staff does not properly report this information to an agency. So, for example, if there is an agency serving 100 individuals across 25 group homes or community-based facilities there could be over 100 staff providing support at one time or another during the week. Some of these staff could have intimate knowledge and experience dealing with an individual with disabilities but others could be new on the job or new to that location or just not as proficient at parts of their job.

There are several problems which could occur. The staff member might report something in one location in the system and not realize they need to file certain forms. For example, a staff member might write a daily log note explaining something which happened but not realize they also have to document that same event in a second location as say an injury or incident report. Currently agencies and states may have human beings checking that reports match. This can be difficult to do and especially difficult to do in real time to meet state reporting requirements as well try to take action to improve care. One aspect of this system is to match written reports and written information with other required information and either make sure other reports are submitted or prompt when additional reporting and analysis is needed.

Another problem is that a staff member might have written something in a difficult to read manner. This could be on account of them not understanding what was really happening with an individual. This could be that they came from a different culture and they use different language or have different views of similar events. An aspect of this system could be to automatically determine the intent of a staff or written to make sure that the intent and information is properly written in a compliant format required by HIPAA-type regulations.

What is significant here is that it would not be possible to check on this type of reporting mistakes or reporting problems without a new type of analysis and system. The current system that agencies use has left open many reporting errors over time. It would be extraordinarily expensive and probably impossible in many instances to throw enough people at reviewing documentation to solve these problems.

A significant part of the problem is that there is a major workforce crisis for people working with individuals with developmental disabilities or receiving home and community-based services. Even people who are well-intentioned can either make mistakes, not have full information, not be properly trained or take other actions which may be problematic. Historically front-line staff are among the lowest paid workers in the communities they live and work in. There is a significant turnover in jobs, which means many staff may not have experience working with the individuals they are working with, and if they do, may not have exposure and ability to factor in other data to their analysis or reporting.

While there is no typical background for direct support professionals (“DSPs”) who are often working with individuals on a three-shift basis, seven days a week, there are certain common expectations. Words like empathy, compassion, desire to help others, patience, flexibility, and caring are typical traits for these staff. However, many staff have limited to no experience working in the field. Many have a GED degree or limited college. Most staff do not have 4-year college degrees. Associate degrees and certificates are more common. While this may provide caring staff who want to do the right thing for the individuals they provide service and supports to, it can be problematic as there is more data, more reporting and regulations, and more need to combine information from different data sources to figure out the best approaches to support an individual as well as properly report on what is happening for regulatory and agency requirements, including HIPAA-type regulations.

To compound the problem there is a significant turnover among people providing supports to people receiving HCBS services. This means that training is often limited. It also means that knowing the full story, goals, objectives, medications, and reactions of a person is limited in many cases due to the short history of the staff in this field.

So, the current process in some government agencies may be to have humans read all daily log notes or all of certain types of documentation and see if they match filed incident reports. The human surveyors or reviewers are limited in what they can review and compare. They might not be able to check on significant amounts of medications, sleep data, staffing data, video data and other types of information which might indicate that some sort of event was not properly reported. This is very time-consuming and not fully accurate or timely.

The current/historical system of people reading daily notes, behavioral reports, and other documents, and then determining whether something meets a threshold of a reportable incident cannot be done in a timely and consistent manner by even the best-trained staff. But it is a particular problem for poorly trained or inexperienced staff. Even staff who have been working for years at an agency might not be familiar with the particular needs or conditions of a specific individual they are reviewing information about. There could be new information about a doctor's visit or a medical condition or a family situation which changed even for an individual a staff has been dealing with for a while.

It is also possible that there is information that no human, guardian, or staff is aware of about an individual because it was unobserved by people. But this information could have been documented by a sensor or a camera or other device automatically capturing information. In this instance a computer system could make a decision on the need to document or take action about a situation in a manner that no individual could.

There is a particular need for organizations funded by or through organizations such as Health and Human Services or individual state departments of developmental disabilities or departments of aging or other Home and Community Based Supports (“HCBS”) organizations including insurance companies and managed care organizations. These funding organizations often have requirements to document certain activities including injuries, medication errors, hospitalizations, falls, and other situations or events which may vary by jurisdiction, funding source, agency, state or other oversight or funding organization.

As computers become more powerful there are different type of approaches to getting the information needed. From the perspective of an individual receiving home and community-based services they do not care if their recommendations and reporting is created by algorithms, artificial intelligence, human observation, or other tools which may exist today or may need to be created.

Some of these issues and concerns are addressed in the Federal Register Final Rule https://www.federalregister.gov/documents/2024/05/10/2024-08363/medicaid-program-ensuring-access-to-medicaid-services from the Centers for Medicare & Medicaid Services (“CMS”), Department of Health and Human Services (“HHS”). The regulations were effective on Jul. 9, 2024. The federal summary of the rule was “This final rule takes a comprehensive approach to improving access to care, quality and health outcomes, and better addressing health equity issues in the Medicaid program across fee-for-service (FFS), managed care delivery systems, and in home and community-based services (HCBS) programs. These improvements increase transparency and accountability, standardize data and monitoring, and create opportunities for States to promote active beneficiary engagement in their Medicaid programs, with the goal of improving access to care.” The purpose of including some of these excerpts is several-fold. One is to show this is a significant problem which has not been able to be addressed in the past. “The Medicaid program provides essential health coverage to tens of millions of people, covering a broad array of health benefits and services critical to underserved populations, including low-income adults, children, parents, pregnant individuals, older adults, and people with disabilities.” This is a costly problem which has not been able to be solved by the federal and state governments.

The final rule in the Federal Register states “Some of the most common feedback we received through the RFI related to ways that we can promote health equity through cultural competency. Commenters shared the importance that cultural competency plays in how beneficiaries access health care and in the quality of health services received by beneficiaries. The RFI respondents shared examples of actions that we could take, including collecting and analyzing health outcomes data by sociodemographic categories; establishing minimum standards for how States serve communities in ways that address cultural competency and language preferences; and reducing barriers to enrollment and retention for racial and ethnic minority groups.”

Georgetown University Health Policy Institute (https://hpi.georgetown.edu/cultural/) defines cultural competency in health care as “Cultural competence is defined as the ability of providers and organizations to effectively deliver health care services that meet the social, cultural, and linguistic needs of patients. (1) A culturally competent health care system can help improve health outcomes and quality of care, and can contribute to the elimination of racial and ethnic health disparities. Examples of strategies to move the health care system towards these goals include providing relevant training on cultural competence and cross-cultural issues to health professionals and creating policies that reduce administrative and linguistic barriers to patient care.” The definition basically focuses on “training” and “policies” to reduce barriers. These are outdated concepts in the world of using person-centered data within a large data-driven real time system.

Given the complex needs of individuals who in many cases have significant cognitive or other disabilities which limit their ability to communicate, this concept of focusing on training staff or training people as a primary manner of understanding goals, needs, outcomes, and objectives is problematic and is the disclosed inventions are designed to solve this problem. For example, a staff member could write or verbally report a daily log note or other periodic report which is limited in scope based on the education, experience, or cultural background of the staff member. A real time system could be designed to flag that note either to a supervisor, an oversight agency or even back to the staff themselves to check for something specific which might be missing.

The current human-monitored system is not a real-time system. So, reports or misaligned data might not be reviewed for hours, days or even weeks after something has occurred. So, while the current human-monitored and checked system might file a timely report, it could miss an opportunity to catch a need for hospitalization, prevent an abuse and neglect situation, adjust medications, or take other actions as appropriate.

For example, if three different staff members at day programs working with three different individuals wrote daily log notes reporting different problems with those individuals in a given day, the problem could have been that there was a problem with the overnight staff at a residential facility. The overnight staff could have been new and having a problem giving medications. The overnight staff could have misunderstood instructions regarding bed monitoring at night and caused the three individuals to have had a bad night's sleep. There are many other examples where current monitoring would not catch problems before they escalated into something more significant. And the new proposed regulations are not really thinking or planning for real-time suggestions and monitoring.

The Final Rule confirms these concerns “In addition, although there are differences in rates of disability among demographic groups, there are very limited data currently available to assess disparities in HCBS access, utilization, quality, and outcomes. Few States have the data infrastructure to systematically or routinely report data that can be used to assess whether disparities exist in HCBS programs. This lack of available data also prevents CMS and States from implementing interventions to make improvements in HCBS programs designed to consistently meet the needs of all beneficiaries. Compounding these concerns have been notable and high-profile instances of abuse and neglect in recent years, which have been shown to result from poor quality care and inadequate oversight of HCBS in Medicaid.”

The Federal Register acknowledges that there is a problem in just relying on current training and policies to solve this problem. It quoted a 2018 report, “Ensuring Beneficiary Health and Safety in Group Homes Through State Implementation of Comprehensive Compliance Oversight,” (“Joint Report”), which was jointly developed by the U.S. Department of Health Human Services'Administration for Community Living (ACL), Office for Civil Rights (OCR), and the Office of Inspector General (OIG), found systemic problems with health and safety policies and procedures being followed in group homes and that failure to comply with these policies and procedures left beneficiaries in group homes at risk of serious harm.”

A significant part of the problem is staff turnover and the workforce crisis in this field. According to the Medicaid and CHIP Payment and Access Commission “High rates of turnover driven by low wages, lack of advancement opportunities, and worker dissatisfaction all contribute to shortages of HCBS workers. According to PHI, HCBS workers have a turnover rate of 40 to 60 percent annually (PHI 2021)” https://www.macpac.gov/wp-content/uploads/2022/03/MACPAC-brief-on-HCBS-workforce.pdf. That article and others give many reasons for the challenges with retaining experienced workers including training, career opportunities, wages and more. But having non-experienced staff with limited training writing up events and occurrences is leading to inconsistencies in reporting and a lack of ability to take proper real-time actions. The problem of reporting is still a challenge for staff with experience because of the vase amount of knowledge required to make proper decisions. People should not be solely making these decisions when AI, ML, algorithms, and other systems are available to help. The staff turnover also leads to more citations at agencies and more costs for agencies and government and other oversight agencies to hand monitor the situation, which is basically impossible to possibly factor in all the varied combinations of data.

There is an interesting issue that the government is looking at the Access Monitoring Review Process (“AMRP”) in a somewhat backwards-looking manner. Beyond the question of whether this is the right process, what is interesting and informative is the type of process the government is considering in the most recent 2024 Final Rule. The Final Rule almost complains that “The unstandardized nature of the AMRPs, which largely defer to States to determine appropriate data measures to review and monitor when documenting access to care, have made it difficult to assess whether any single State's analysis demonstrates compliance with section 1902(a)(30)(A) of the Act.”

### SUMMARY OF THE INVENTION

The Federal Register Final Rule, and the other reports cited above, do not propose solutions to these problems. The disclosed embodiments of this invention include systems and methods for solving some of these problems.

The federal government is looking to create a system where they hope to take action about collecting and analyzing health outcomes data and then establish minimum standards for future regulations. The intent of the disclosed invention is to go beyond what the government is thinking about. The disclosed system takes real time data and combines with advanced analytics including algorithms, machine learning, artificial intelligence and more that can look at combinations of person specific data, staffing information, agency specific information, government regulations, outside medical information and more, and take that information and provide real-time suggestions and actions, including escalating review, to improve the lives of individuals. The system would be able to adjust based on real information in its system factoring in cultural competency.

In particular, the disclosed embodiments of this invention deal with unstandardized data in novel ways by training and using large language models (“LLMs”) from unstructured data and/or combinations of structured and unstructured data.

Aspects of the present invention also provide a system for managing actions and documentation and other information from individuals under care among multiple security domains and caseloads. These individuals can include those with cognitive disabilities, prisoners, children in school, infants aged three to three, the elderly, and others. Each of the systems and methods disclosed in compliance with Personal Health Information (“PHI”) across and within organizations in the human service field in compliance with HIPAA-type regulations, as well as security procedures and objectives of furthering person-centered-thinking.

Embodiments of this system are built upon the concept of caseloads and roles previously described in the '370 Patent at Col. 5, Line 21-Col. 7, Line 32; Col. 12, Line 1-Col. 13, Line 8; Col. 22, Lines 30-59, Col. 24, Lines 40-57 (caseloads) and in the '370 Patent at Col. 13, Line 4-Col. 15, Line 10; Col. 22, Lines 30-59 (roles and super roles).

The disclosed systems and methods are intended to automatically perform the analyses required by HIPAA-type regulations and other requirements described above, and more. It is intended to be more accurate, at a lower cost, and timelier. And it performs tasks and provides information and support to people with cognitive and other needs who would not have been able to have access to that support.

As noted above, it's difficult if not impossible to get consistent reviews applied across a collection of human-created writeups. A mechanized system would provide more uniform and consistent reviews than a collection of human reviewers.

The above citation of government reports, studies, commentary, and regulations shows that the disclosed methods and systems are not replacing what people have historically done or even what many agencies are currently thinking should be done. These are novel approaches which add new conceptual and technical ways or solving problems which have not been able to address previously. The disclosed combinations of software, hardware, sensors, human training, and other systems and methods allows better care, better compliance with HIPAA-type regulations and objectives in a structure and cost which is manageable.

There are many quotes about engineers solving problems that people didn't know they had. To some extent the goal of this invention is to solve problems people and governments should realize they have, but are not even thinking about trying to solve, and do not have ways of addressing them.

Artificial Intelligence/machine learning is used to improve HIPAA-compliant computer systems and methods for linking electronic GER and T-Log records relating to individuals under care by a caregiver. T-log records are compared to GER subject matters to determine whether an accuracy score exceeds a threshold. If so, the T-Log record is compared to GER events to determine whether an accuracy score exceeds a threshold. If so, the T-Log record is compared to GER subtypes to determine whether an accuracy score exceeds a threshold. If so, the T-Log record is compared to existing GER records to determine whether an accuracy score exceeds a threshold. If so, the T-Log and GER records are electronically linked and an alert is provided to the caregiver. One or more of these comparisons is performed at least in part by categorizing the T-Log record by using the one or more large language models.

Systems and devices that perform the functions at least of: (1) storing records of events relating to one or more individuals under care; (2) using an LLM AI model to review these records; and (3) based on the review, providing suggestions and/or actions including escalating review, were and are neither routine, well-understood, nor conventional in the field of logging and reporting of events for individuals under care.

Systems and devices that perform the functions at least of: (1) providing a database of stored GER subject matters; (2) providing a database of stored GER events; (3) providing a database of stored GER subtypes; (4) providing a database of stored existing GER records; (5) providing a device for use by the caregiver; (6) providing a link database for storing links between GER records and T-Log records; (7) storing a GER subject matter threshold; (8) storing a GER event threshold; (9) storing a

GER subtype threshold; (10) storing at least one GER record relating to the individual; (11) storing at least one T-Log record relating to the individual; (12) comparing the T-Log record to the GER subject matters to determine a match, including measuring a GER subject matter score relating to an accuracy of the match; (13) determining whether the GER subject matter score exceeds the stored GER subject matter threshold; (14) comparing, if the subject matter score exceeds the stored GER subject matter threshold, the T-Log record to the GER events to determine a match, including measuring a GER event score relating to an accuracy of the match; (15) determining whether the GER event score exceeds the stored GER event threshold; (16) comparing, if the subject matter score exceeds the stored GER event threshold, the T-Log record to the GER subtypes to determine a match, including measuring a GER subtype score relating to an accuracy of the match; (17) determining whether the GER subtype score exceeds the stored GER subtype threshold; (18) comparing, if the GER subtype score exceeds the stored GER subtype score threshold, the T-Log record to the existing GER records to determine a match, including measuring an existing GER score relating to an accuracy of the match; (19) determining whether the existing GER score exceeds the stored existing GER threshold; (20) storing, if the existing GER score exceeds the stored existing GER threshold, an electronic link from the T-Log record to the GER record in the link database; and (21) providing a notification of the link to the device, were and are neither routine, well-understood, nor conventional in the field of logging and reporting of events for individuals under care.

In such systems and methods, the functions of: (1) providing a database storing at least one authorization profile associated with the caregiver, wherein the caregiver is associated with one or more roles and one or more caseloads and the caseloads include access privilege information for the individual, wherein the access privilege information included in the caseload includes the identities of individuals to which the caregiver has access; (2) comparing the identity of the individual to the caregiver's authorization profile information, including comparing the identity of the individual to the caregiver's caseload; and (3) providing the notification of the link to the caregiver if the identity of the individual is stored in the caregiver's caseload, were and are neither routine, well-understood, nor conventional in the field of logging and reporting of events for individuals under care.

Furthermore, such system and methods that performed one or more of the functions of comparing the T-Log record to the GER subject matters, comparing the T-Log record to the GER events, comparing the T-Log record to the GER subtypes, and comparing the T-Log record to the existing GER records, at least in part by categorizing the T-Log record by using one or more large language models, were and are neither routine, well-understood, nor conventional in the field of logging and reporting of events for individuals under care.

### BRIEF DESCRIPTION OF DRAWINGS

FIG. 1 illustrates the overall system and its infrastructure.

FIG. 1A illustrates the API Server Pool Process Flow.

FIG. 2A illustrates the workflow of How the QA Assistance Works.

FIG. 2B illustrates the workflow of DSP Notification and Action.

FIG. 2C illustrates the workflow of Supervisor Oversight and Follow-Up.

FIG. 3A illustrates the Dashboard view of QA Assistant.

FIG. 3B illustrates the ‘QA Assistant Dashboard’ search criteria page.

FIG. 3C illustrates a validation message for date ranges exceeding 60 days or for future dates.

FIG. 3D shows the generated QA Assistant Dashboard with provider name, search parameters, and time zone.

FIG. 3E illustrates clicking on Resolved option to open the QA Assistant Data List.

FIG. 3F illustrates the ‘QA Assistant Data List’ accessed from the ‘Resolved’ row of the ‘Action’ criteria.

FIG. 3G illustrates how to filter the dashboard by clicking the Filter icon.

FIG. 3H illustrates selecting options in the Filter Dashboard popup filters the main dashboard.

FIG. 4A illustrates clicking on Open option to open the QA Assistant Data List.

FIG. 4B illustrates a ‘QA Assistant Data List’ accessed from the ‘Open’ row of the ‘Action’ criteria.

FIG. 4C illustrates clicking the “Create New GER” button to create a GER from the recorded T-Log.

FIG. 4D illustrates creating a GER.

FIG. 4E illustrates Submitting a GER.

FIG. 4F illustrates clicking the “Not a GER” button in the Action column to confirm that the recorded T-Log is not a GER.

FIG. 4G illustrates a success message stating the T-Log has been changed to ‘Not Relevant’.

FIG. 4H illustrates that to link a T-Log with the matching GER, the user clicks on the Link GER button.

FIG. 4I illustrates a success message stating the T-Log has been changed to ‘Resolved’.

FIG. 5A illustrates a GER form.

FIG. 5B illustrates the dropdown options of the GER form.

FIG. 6A illustrates a T-Log form.

FIG. 6B illustrates the dropdown of a T-Log form.

FIG. 7 illustrates the QA Assistant Process Flow.

FIG. 7A Illustrates the QAA Process Flow (Triggered from GER Side).

FIG. 8 illustrates the QA Assistant Feedback and Incremental Model Update Pipeline Diagram.

FIG. 9A illustrates the QA Assistant Dashboard view from Agency Repots tab.

FIG. 9B illustrates the QA Assistant Dashboard from Individual tab.

FIG. 9C illustrates the ‘QA Assistant Dashboard’ search criteria page.

FIG. 9D illustrates a validation message for date ranges exceeding 60 days or for future dates.

FIG. 9E illustrates a validation message for date cannot be later than today's date.

FIG. 9F illustrates the generated QA Assistant Dashboard with provider name, search parameters, and time zone.

FIG. 9G illustrates clicking on Resolved option to open the QA Assistant Dashboard.

FIG. 9H illustrates the ‘QA Assistant Dashboard’ accessed from the ‘Resolved’ row of the ‘Action’ criteria.

FIG. 9I illustrates how to filter the dashboard by clicking the Filter icon.

FIG. 9J illustrates selecting options in the Filter Dashboard popup filters the main dashboard.

FIG. 10A illustrates clicking on Open option to open the QA Assistant Dashboard.

FIG. 10B illustrates a ‘QA Assistant Dashboard’ accessed from the ‘Open’ row of the ‘Action’ criteria.

FIG. 10C illustrates clicking the “Create New GER” button to create a GER from the recorded T-Log.

FIG. 10D illustrates creating a GER.

FIG. 10E illustrates Submitting a GER

FIG. 10F illustrates clicking the “Not a GER” button in the Action column to confirm that the recorded T-Log is not a GER.

FIG. 10G illustrates a success message stating Not a GER action is successful.

FIG. 10H illustrates that to link a T-Log with the matching GER, the user clicks on the Link GER button.

FIG. 10I illustrates a success message stating the T-Log has been changed to ‘Resolved’.

FIG. 10J illustrates clicking on the Enter Vital Signs button to create Vital signs.

FIG. 10K illustrates opening the new Vital Sings form 1011.

FIG. 10L illustrates submitting the Vital Signs form.

FIG. 10M illustrates the error message for those who doesn't have sufficient privilege.

FIG. 10N illustrates clicking the “Not a GER” button under the action column if the T-Log does not concern any Vital Signs

FIG. 10O illustrates the status of the QAA form will change from ‘Open’ to ‘Dismissed’ if the T-Log does not concern Vital Signs.

FIG. 11 illustrates a notification popup sent to a user when QA Assistant detects a T-Log.

FIG. 12A illustrates the QA Assistant floating sidebar in the dashboard.

FIG. 12B illustrates the QA Assistant side panel with Open and Resolved tabs.

FIG. 12C illustrates the clicking on the Mark As Seen button.

FIG. 12D illustrates clicking the Mark All As Seen button to remove the highlight from all notifications in the Open tab.

FIG. 12E illustrates clicking on the Create GER button from the QA Assistant side panel.

FIG. 12F illustrates creating a new GER.

FIG. 12G illustrates clicking on the Enter Vital Signs button from the QA Assistant side panel.

FIG. 12H illustrates creating a new Vital Signs form.

FIG. 12I illustrates clicking on the Link GER button from the QA Assistant side panel.

FIG. 12J illustrates navigating to the Resolved tab to locate the T-log that is Linked to GER.

FIG. 12K illustrates clicking on the Not a GER or Not Vital Signs button from the QA Assistant side panel.

FIG. 12L illustrates opening a feedback popup window.

FIG. 12M illustrates clicking on the Add a Feedback button.

FIG. 12N illustrates Provide your Feedback popup window where user can provide their feedback.

### DETAILED DESCRIPTION OF THE INVENTION

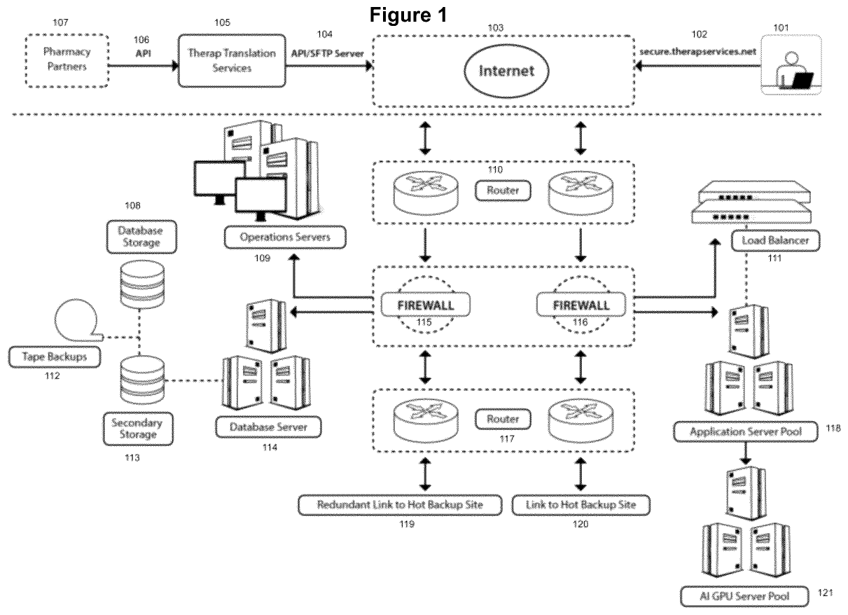

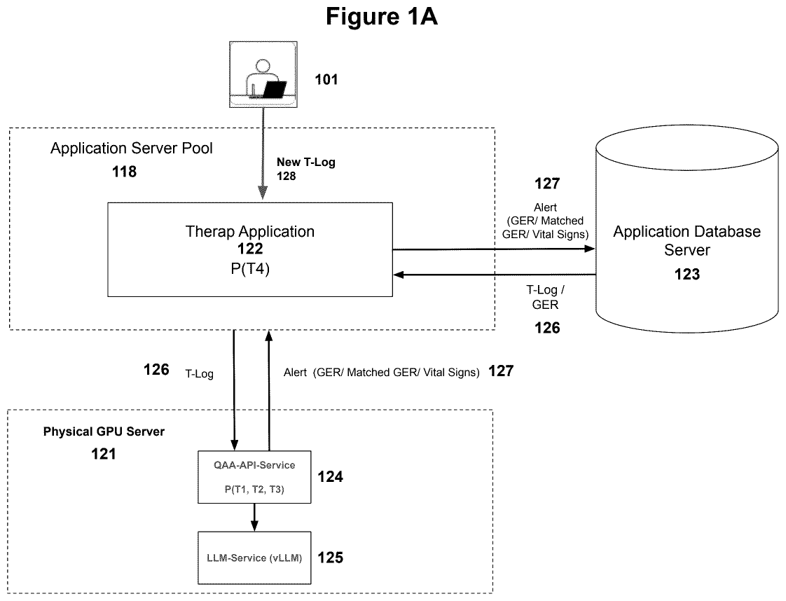

FIG. 1 illustrates a preferred embodiment of the overall system and its infrastructure. The infrastructure manages data flow from various sources through a structured and secure path. Users 101 connect to the system via the Internet 103 using the secure.therapservices.net 102, while Pharmacy Partners 107 send data through an API 106 to Therap Translation Services 105, which then forwards the data to the Internet 103 via an API/SFTP 104 server. Data from the Internet 103 first passes through a Router 110 and is then processed by two Firewalls, 115 and 116. Data going through Firewall 115 is directed to the Operations Servers 109 and the Database Server 114, which in turn connects to the primary Database Storage 108, Secondary Storage 113, and Tape Backups 112. In parallel, traffic passing through Firewall 116 is sent to a Load Balancer 111 that distributes requests to the Application Server Pool 118. From Application Server Pool 118, data goes to AI GPU Server Pool 121. From Firewall 115 the data goes back and forth to Router 117 and from there the data goes back and forth to Redundant Link to Hot Backup Site 119. Similarly from Firewall 116 the data goes back and forth to Router 117 and from there the data goes back and forth to Link to Hot Backup Site 119.

FIG. 1A illustrates the service level architecture of the QA Assistant module. The implementation follows a client-server architecture where the QAA Service 124 in physical GPU Server 121 provides the model capabilities to identify significant events like T-Log 126 from a given text, and the Therap application uses this service to identify potential GER events 127 within a T-Log description. As the Therap Application 122 is working as a client in Application Server Pool 118, whenever a new T-Log 128 is created, the Therap Application 122 sends the T-Log description 126 to the QAA service 124 and gets the event label (GER/Non-GER/Vital Signs) 127 as a response. Additionally, for the T-Logs 126 alerted by the QA Assistant, a cross-matching operation occurs with the existing GERs, or whenever a new GER is created, to ensure that the alerted event has been reported in the GER module. This cross-matching operation is also initiated by the Therap Application 122 and executed by the QAA Service 124. A daily Cron at the application side uses the LLM Service 125 to identify T-Logs that were mislabeled by the primary models of the QA Assistant. These stats on mislabeled data is collected as part of the incremental update process of the QA Assistant models to achieve better accuracy. All the data used as a source, or produced by the QAA/LLM services are stored in the Therap Application Database Server 123, the QAA/LLM services do not store any generated alerts/responses.

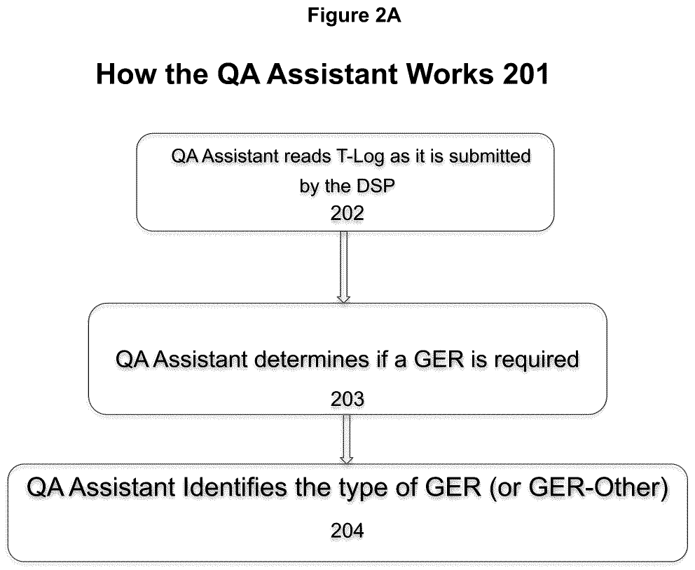

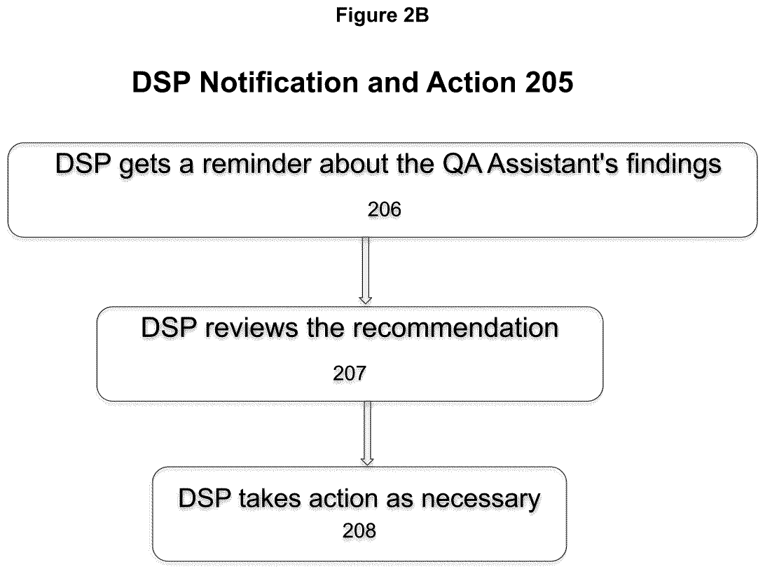

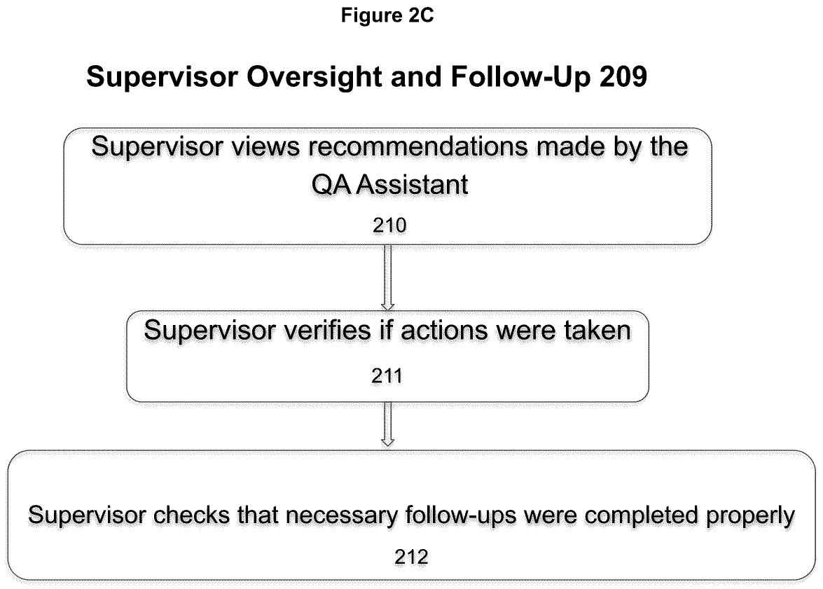

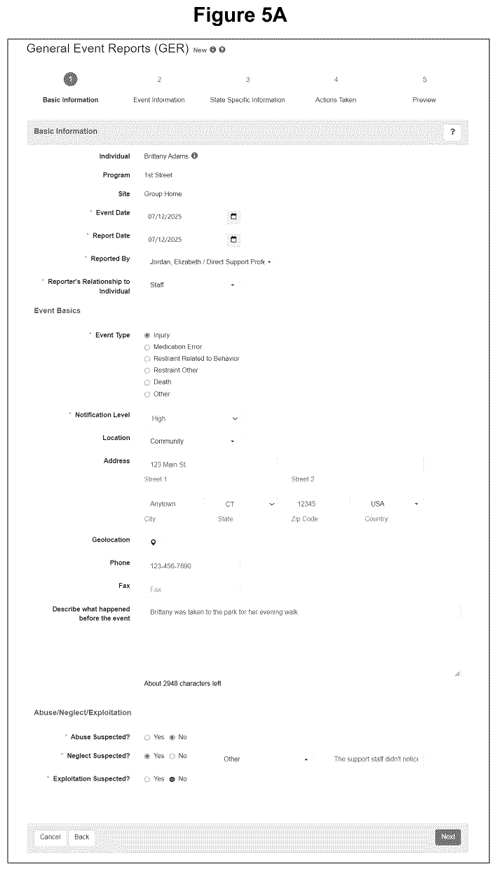

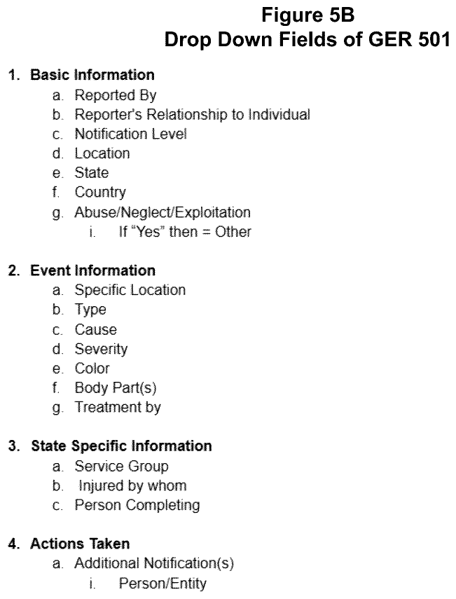

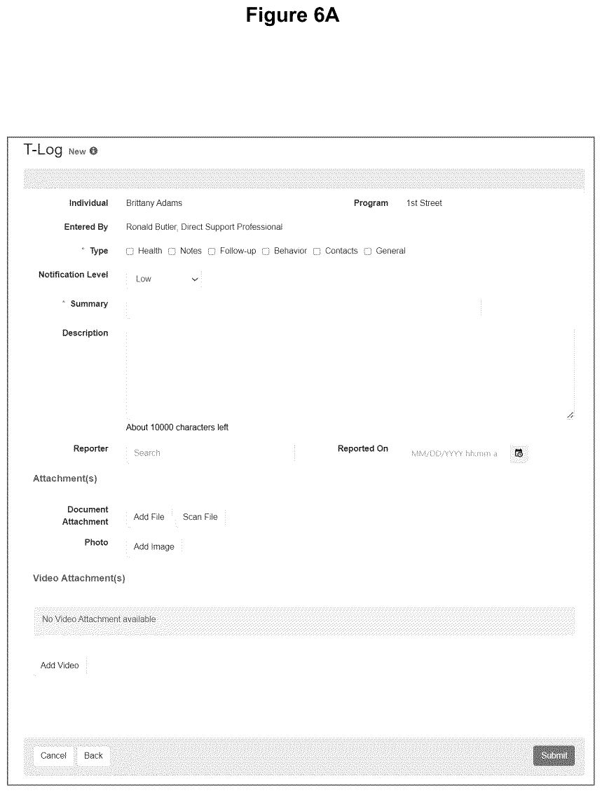

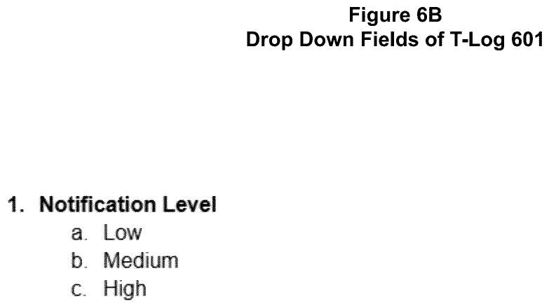

FIG. 2A illustrates the workflow of How the QA Assistance Works 201. In the first step of the workflow the QA Assistant reads the T-Logs 202 (see FIG. 6A and description below) as it is submitted by the DSP. After reading the T-Logs, QA Assistant determines if a General Event Report (GER) (see FIG. 5A and description below) is required 203 or not. Finally if the GER is required then QA Assistant identifies the type of GER (or GER-Other) 204.

FIG. 2B illustrates the workflow of DSP Notification and Action 205. In this workflow, first the DSP gets a reminder about the QA Assistant's findings 206. After that the DSP reviewed the recommendation provided by the QA Assistant 207. Finally DSP takes action if necessary 208.

FIG. 2C illustrates the workflow of Supervisor Oversight and Follow-Up 209. Here the Supervisor views the recommendations 210 that are made by the QA Assistant in the first step. Then the Supervisor verifies if the actions were taken 211 or not. Finally, the Supervisor checks if the necessary follow-ups were completed properly 212 or not.

### QA Assistant Dashboard

Users assigned with the QA Assistant administrative roles (as described in the '370 Patent as referenced above) are able to generate QA Assistant Dashboard for individuals within the agency.

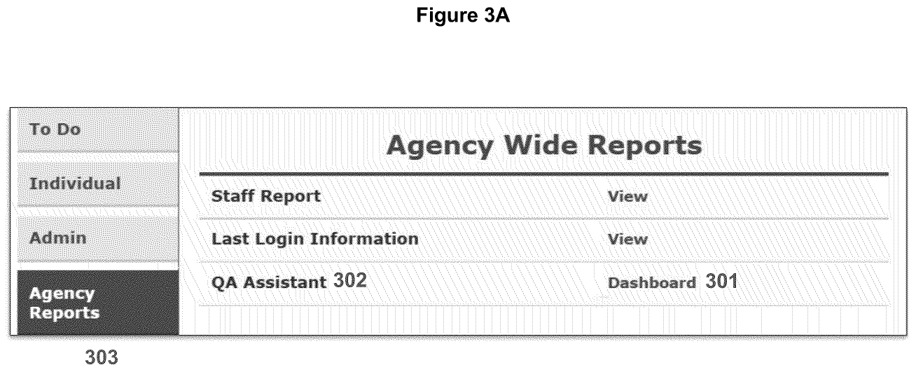

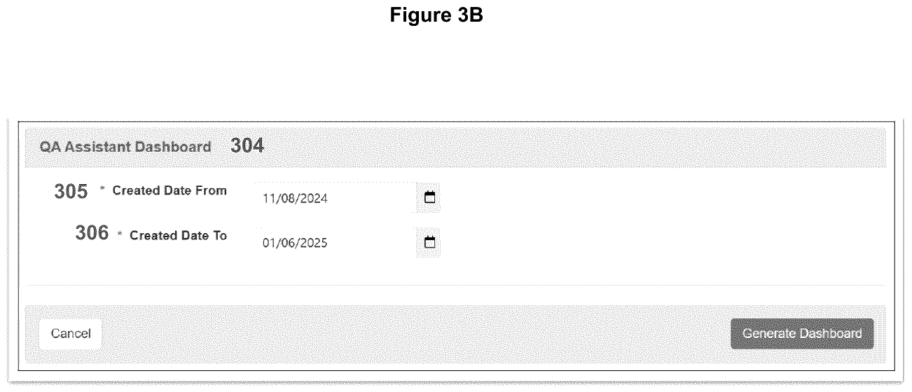

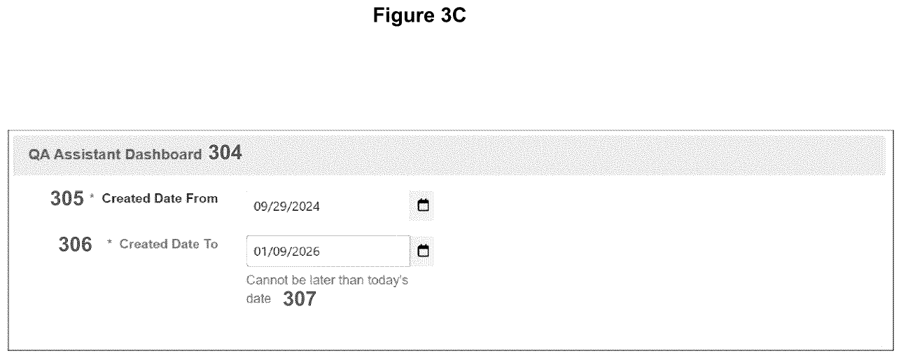

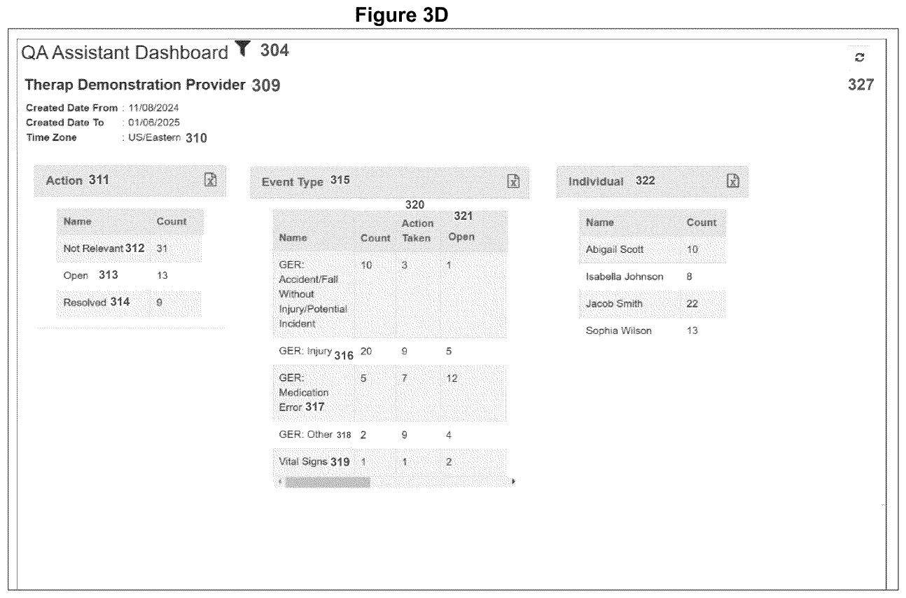

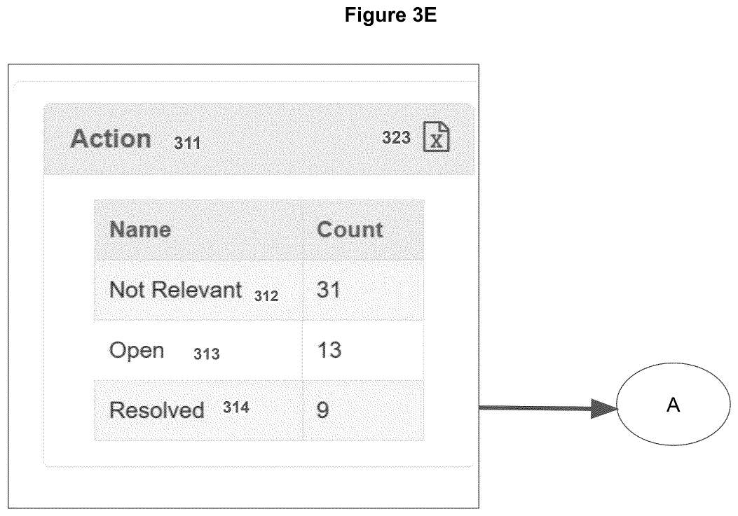

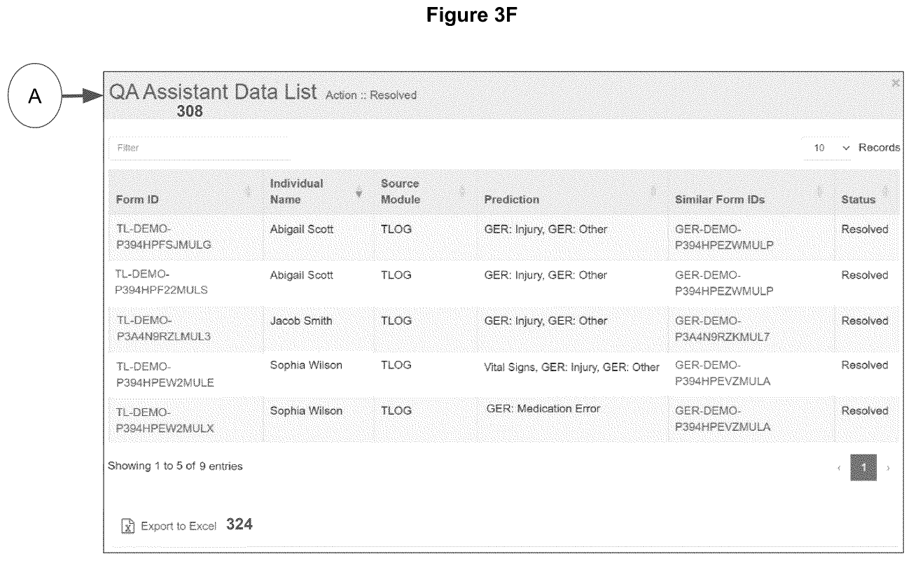

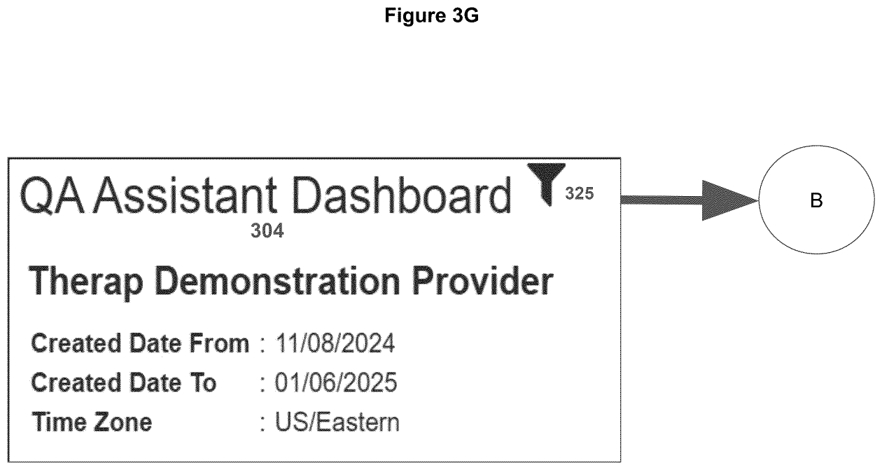

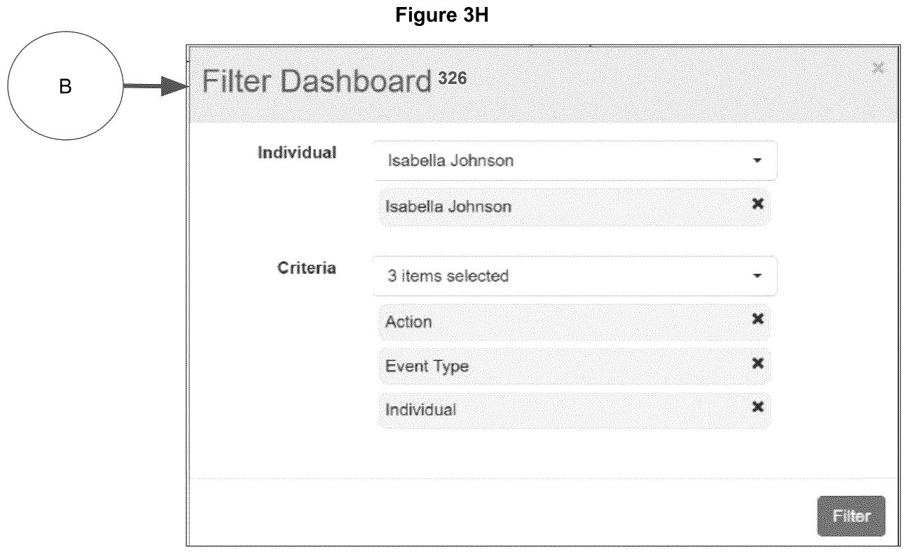

Generating QA Assistant Dashboard: FIGS. 3A-3H show exemplary screen for generating a QA Assistant Dashboard.

FIG. 3A illustrates clicking on the Dashboard 301 link next to the QA Assistant 302 option on an authorized user's Agency Reports tab 303.

FIG. 3B illustrates the ‘QA Assistant Dashboard’ 304 search criteria page. The last 60 days date range is selected in the Created Date from 305 to Created Date To 306 fields, by default. Users can also manually select a different date range within 60 days and then search data using the Generate Dashboard button.

FIG. 3C illustrates if a user searches data for a date range that is beyond 60 days, or selects a date that is later than the current date, the user receives a validation 307. In such cases, the user selects a valid date range, and then re-generate the dashboard.

FIG. 3D illustrates the generated QA Assistant Dashboard 304 that displays the name of the provider 309, the search parameters for which the dashboard was generated, and the time zone 310 selected in the ‘Personal Details’ page of the user who generated the Dashboard. The following criteria sections are available:

Action 311: Based on the actions taken to T-Logs using QA Assistant 302, the following counts are displayed:
a. Not Relevant 312: Lists the T-Logs (See FIG. 6A and description below) for which ‘Not a GER’ or ‘Not a Vital Signs’ was selected from the QA Assistant 302 floating panel.
b. Open 313: Lists the T-Logs that are available in the ‘Open’ 313 tab of the QA Assistant 302.
c. Resolved 314: Lists the T-Logs that are available in the ‘Resolved’ 314 tab of the QA Assistant 302.

Event Type 315: Counts for the following event types are displayed:
a. GER: Injury 316
b. GER: Medication Error 317
c. GER: Other 318
d. GER: Restraint
e. Vital Signs 319

Breakdown of the total T-Log counts are displayed under the Action Taken 320 and Open 321 columns. If an action has been taken to the T-Log they are added under the Actions Taken 320 column, and the T-Logs that have not been resolved 314, are added under the Open 321 column.
a. Individual 322: Lists the individuals whose T-Logs have been considered for potential GERs (see FIG. 5A and description below) and/or Vital Signs.

FIG. 3E illustrates that a user can export the specific list and Excel report by clicking on the Excel icon 323 in the top right corner of each section. Users can click on the Resolved 314 (indicated by A) option to open the QA Assistant Data List 308.

FIG. 3F shows the ‘QA Assistant Data List’ 308 (indicated by A) as described with reference to FIG. 3E and illustrates that clicking on the rows of a section opens up the ‘QA Assistant Data List’ 308 (indicated by A) where the T-Logs that make up the count are displayed. Users can also export the list into excel using the Export to Excel 324 link at the bottom of the popup window. Users with the appropriate caseload-based roles (as described in the '370 Patent as referenced above) are able to click on the T-Log or GER Form ID links to open the respective forms in a new tab.

FIG. 3G illustrates that to narrow down the displaying QA Assistant Dashboard 304, users can click on the Filter icon 325 (indicated by B) on the top of the page.

FIG. 3H shows selecting the appropriate information on the ‘Filter Dashboard’ 326 popup window (indicated by B), and filters the QA Assistant Dashboard 325.

Users can also use the Refresh button 327 on the top right corner of the QA Assistant Dashboard 304 FIG. 3D to reset the searched data.

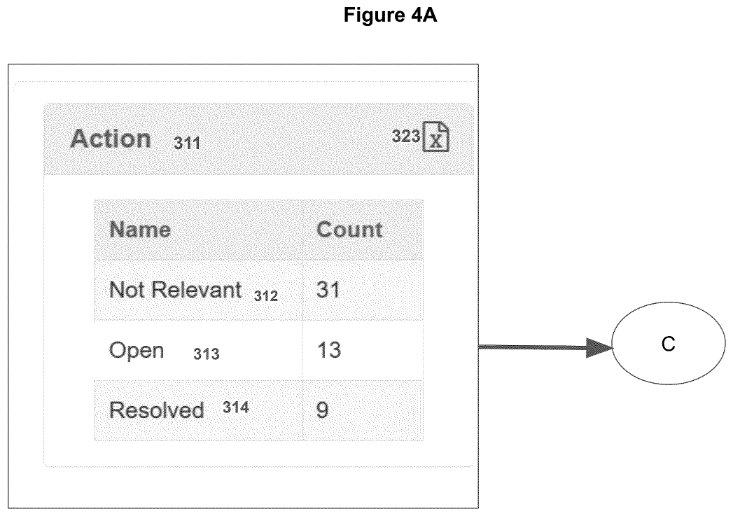

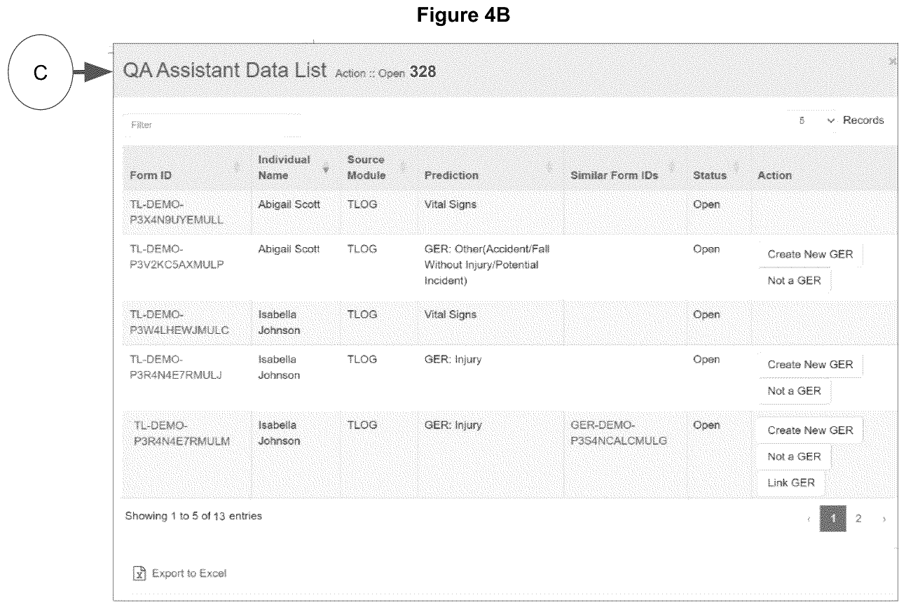

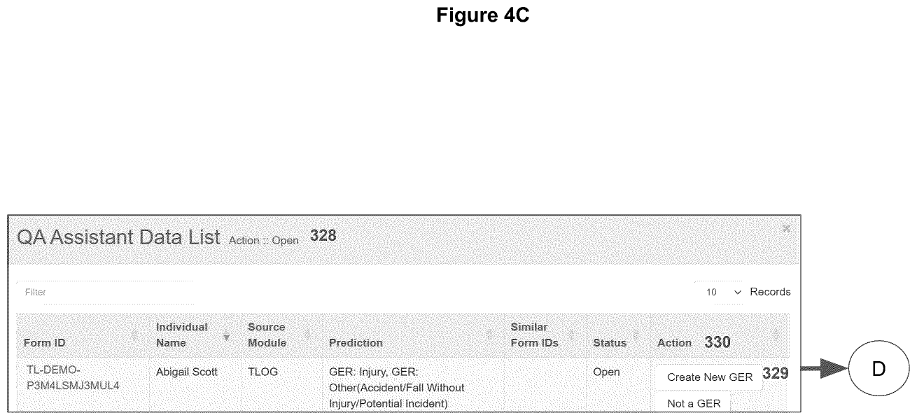

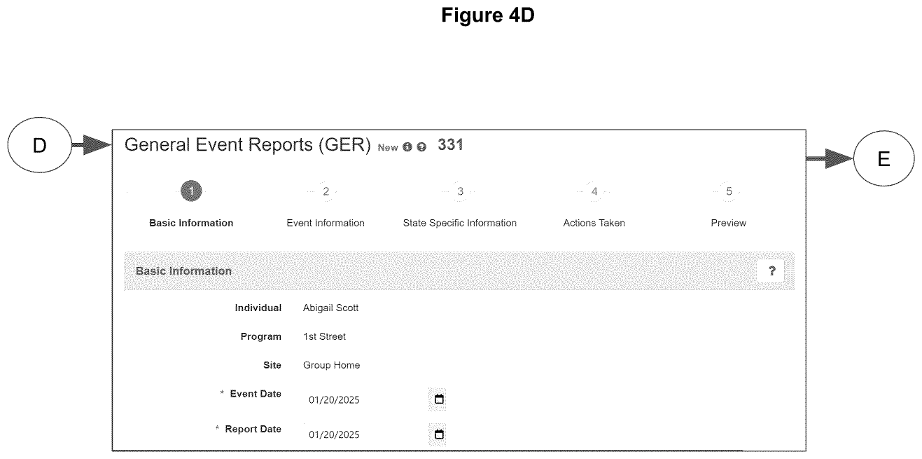

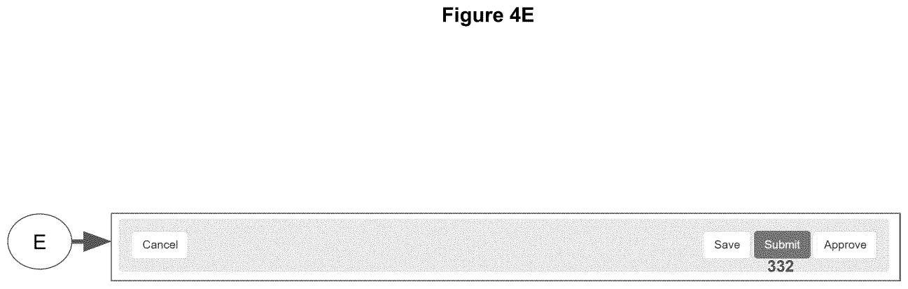

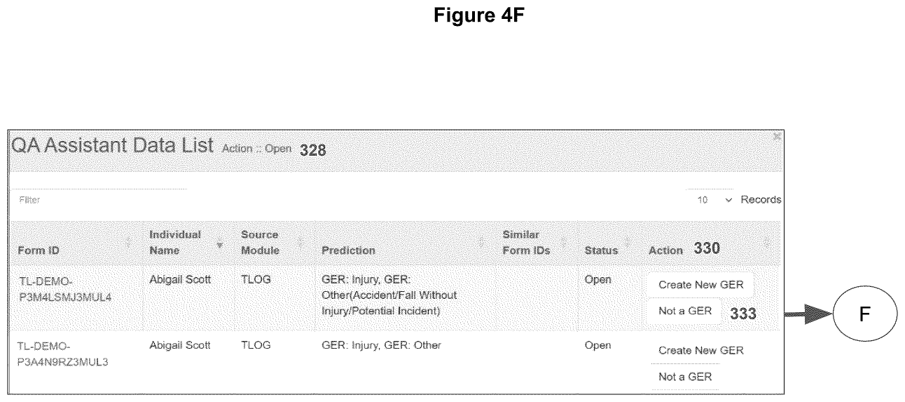

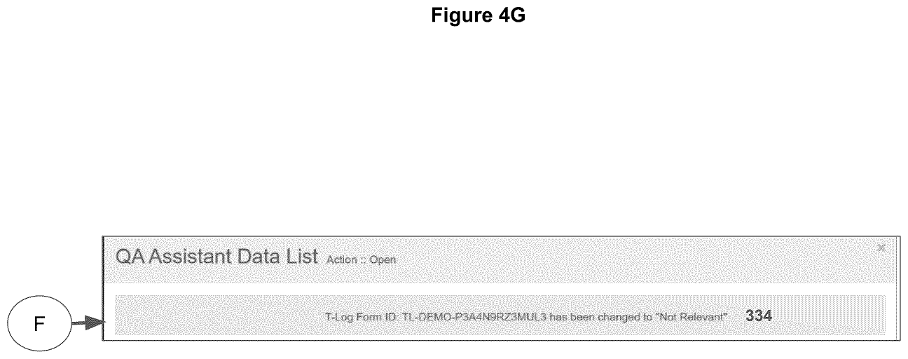

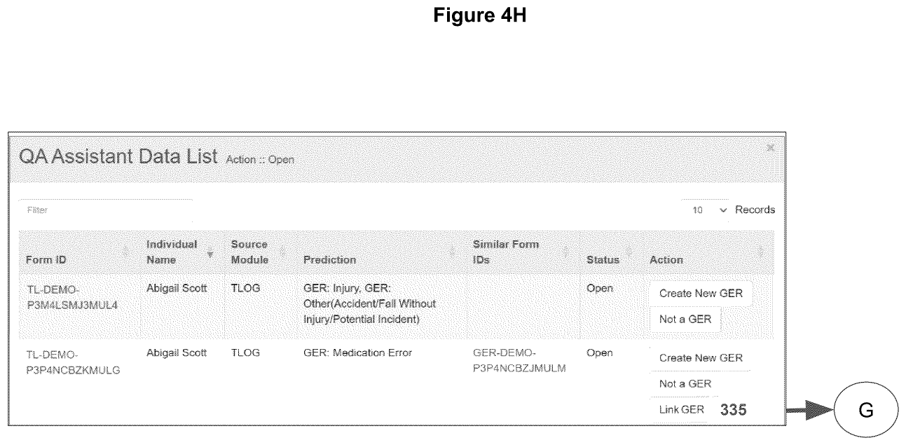

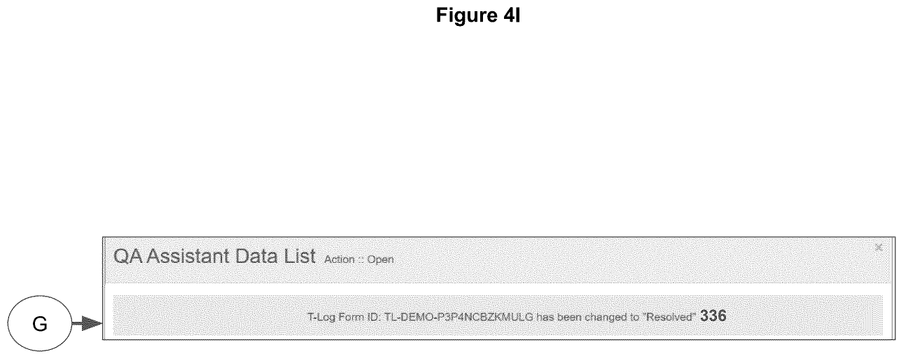

Create and Link GERs from QA Assistant Dashboard: FIGS. 4A-4I show exemplary screen for creating and linking one or more GERs from the QA Assistant Dashboard.

FIG. 4B illustrates a ‘QA Assistant Data List’ 328 (indicated by C) accessed from the ‘Open’ 313 (indicated by C) row of the ‘Action’ 311 criteria FIG. 4A, the type of the event(s) are listed under the Prediction column. If any matching GER is detected, it displays under the Similar Form IDs column.

FIG. 4C illustrates that to create a GER for the recorded T-Log, the user may click on the Create New GER 329 (indicated by D) under the Actions 330 column. The user is then redirected to a new tab with a new GER form 331 FIG. 4D in it to create a GER (indicated by D) and then the user can Submit 332 (indicated by E) the GER, as shown in FIG. 4E.

FIG. 4F shows that to confirm the recorded T-Log is not a GER, the user clicks on the Not a GER 333 button (indicated by F) under the Action 330 column. A success message is displayed on top of the pop-up window (indicated by F), stating that the T-Log has been changed to ‘Not Relevant’ 334 as shown in FIG. 4G.

Users with appropriate caseload-based roles (as described in the '370 Patent as referenced above) are able to click on the T-Log link in the success message to open it in a new tab. Consequently, the count in the ‘Open’ 313 row decreases, while the count in the ‘Not Relevant’ 312 row under the ‘Action’ 311 section increases accordingly.

FIG. 4H shows that to link a T-Log with the matching GER, the user clicks on the Link GER button 335 (indicated by G). A success message is displayed on top of the pop-up window, stating that the T-Log has been changed to ‘Resolved’ 336 (indicated by G) as shown in FIG. 4I.

Users with appropriate caseload-based roles (as described in the '370 Patent as referenced above) are able to click on the T-Log link in the success message to open it in a new tab. Consequently, the count in the ‘Open’ 313 row is decreased, while the count in the ‘Resolved’ 314 row under the ‘Action’ 311 section is increased accordingly.

As discussed above, events and actions taken may be documented in accordance with the preferred embodiments of this invention. Documentation may take the form of General Event Reports or T-Logs, or other formats.

A General Event Report (GER) documents an incident that can include injury, medication error, restraint, death, or the “other” category which may include things such as a person going to the emergency room, etc. Also preferably included within GERs is the opportunity to use a Witness Report to gather further information on a given event from additional staff members who were present.

Preferably, a GER Resolution module is used to document investigation details, recommendations, involved persons and whether or not the investigation is open or closed for the associated GER. Further, a Witness Report preferably gets created if a person is listed as a Witness on the GER and includes the associated GER, Event and Witness details. In addition, a Multi Individual Event module preferably provides users the ability to link related GER forms into one single form, instead of having to document similar GER forms multiple times.

Many states require incident reports to be submitted in a specific format. Preferably, the option to add State-specific information is provided regarding an incident to a GER form. The State-specific GER forms have been designed according to the policy of different States. Preferably, users are also able to take printable version of the State specific information regarding an incident.

FIG. 5A includes preferable GER forms that may be filled in with information generated as part of this invention. In particular, FIG. 5A includes a preferable electronic form format for creating a GER, and FIG. 5B illustrates preferable dropdown fields 501 for receiving information generated as part of this invention

A T-Log preferably offers a simple and effective way for a user (including agencies and their representatives) to enter and share daily shift notes, case notes, contact notes or logs efficiently. A T-Log preferably allows the collection and communication of day-to-day information and progress notes with other staff members who can add follow-ups and share in a way that complies with HIPAA-type regulations.

FIG. 6A includes preferable T-Log forms that may be filled in with information generated as part of this invention. In particular, FIG. 6A includes a preferable electronic form format for creating a T-Log, and FIG. 6B illustrates preferable dropdown fields 601 for receiving information generated as part of this invention.

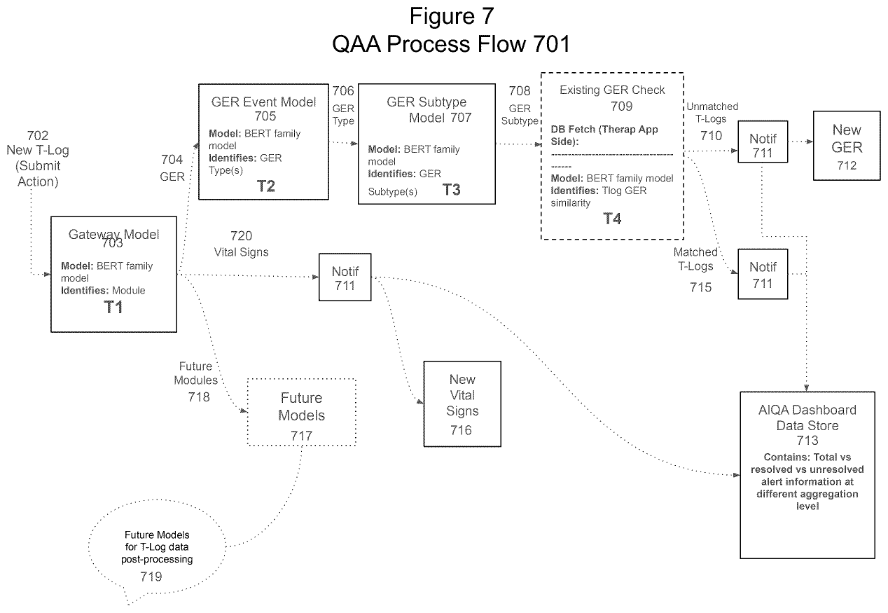

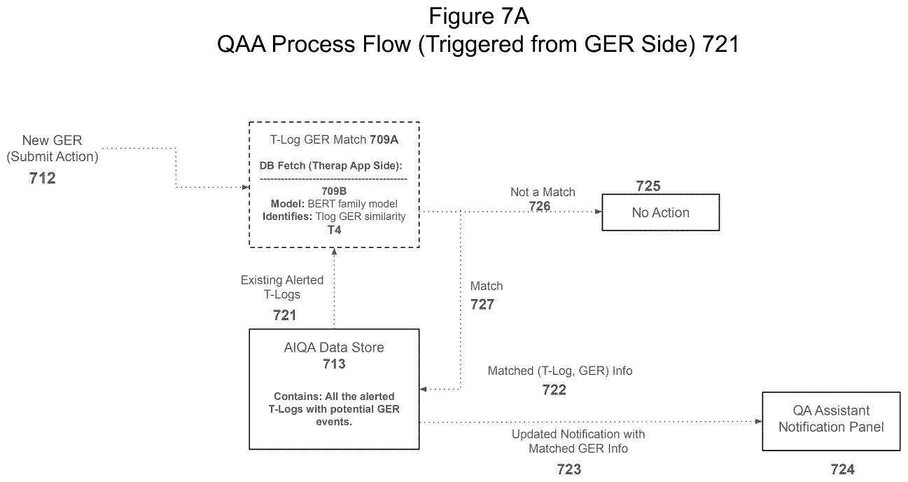

FIG. 7 illustrates the QA Assistant Process Flow 701. This diagram explains how the QA Assistant's notifications are generated “under the hood,” which modules are used for generation, and how it is cross-matched with the GER. The system functions as a Quality Assurance (QA) Assistant automatically analyzing text-based descriptions in T-Logs, identifying key events, cross-matching them with existing records in a GER module, and generating actionable notifications.

Examples of automatic actions that the system may be take based on such notifications are described in the '983 Patent at Col. 30, line 61-Col. 31, line 21, Col. 32, Lines 3-65, Col. 38, line 59-Col. 39, line 3, and FIG. 7, as well as in the '011 Application at ¶¶ 0105, 0163.

As shown in FIG. 7, a BERT family model is used for each of the models used in the preferred embodiment of the system. Leveraging fine-tuning, this family of models excels at multi-label classification tasks required to classify the T-Log, and includes models such as BERTbase, BERTlarge, RoBERTa, DistilBERT, ALBERT, Electra, modernized BERT models such as ModernBERT, and others.

When the Gateway Model 703 infers that the probability of the T-Log 702 referring to a GER 704 exceeds a specified threshold T1, the application passes that T-Log 702, GER 704, to a second model, GER Event Model 705. This model's specific task is to analyze the content and classify it into a predefined GER type 706 with an accompanying probability. The GER 704 system includes a broad category designated as “Other” which contains sub-categories. This third model GER Subtype 707 is invoked only when the preceding GER Event Model 705 classifies the GER type 705 as “Other” with a probability exceeding a specified threshold T2. The GER Subtype Model 707 performs a fine-grained analysis to identify the specific GER Subtype 708 that the T-Log 702 is describing, with a probability exceeding a specified threshold T3. Following the identification, the process enters a cross-matching pipeline, represented by the dotted block in the diagram. This fourth model, the Existing GER Check 709, is responsible for preventing duplicate data entry. It takes the text description of the identified GER type 706 and cross-matches it against the descriptions of previously submitted GERs 704 for that specific individual, with a probability exceeding a specified threshold T4. The objective is to determine if the event described in the T-Log 702 has already been recorded in the GER module.

The final step in the workflow is the generation of Notifications 711 based on the outcome of the cross-matching pipeline. If the system does not find a corresponding GER, it issues a Notification 711 to the user having authorized access to a specific individual's data as permitted by the user's caseload(s) and role(s) (as described in the '370 Patent as referenced above). This Notification 711 suggests that the New GER 712 should be created for the event identified in the T-Log 702. If a potentially matching GER is found, the system generates a Notification 711 informing the authorized user of a potential GER event detected in the T-Log 702. Alerts and Notifications 711 generated by the system are stored in an AIQA Dashboard Data Store 713. This data is then served to authorized users through a QA Assistant Dashboard 304. This dashboard allows authorized users to monitor the system's output, providing a clear view of total number of alerts generated for a specific Individual or provider over a period and the status of each alert, indicating whether it has been actioned. The alerts are categorized into three distinct states:
Open 911: The initial state of an alert, indicating that no action has yet been taken.
Resolved 912: The user has addressed the alert, typically by linking the T-Log to a new or existing GER.
Dismissed/Unresolved 913: The user has reviewed the alert and manually discarded it, deeming it irrelevant.

The system is designed to handle more than just GERs 704. The Gateway Model 703 can also identify other types of events. If the Gateway Model 703 identifies content related to Vital Signs 720, it triggers a secondary workflow. A Notification 711 is generated informing the user that Vital Signs 720 data has been mentioned in the T-Log 702 and suggests creating a new Vital Signs 716 entry.

Currently, no further processing or cross-matching is performed on Vital Signs 720 data, but the architecture allows for this functionality to be added in the future. These alerts are also stored in the AIQA Dashboard Data Store 713 and visible on the dashboard. The Gateway Model 703 acts as a central router that can be expanded to identify new categories of information over time. For instance, if a Future Modules 718 for identifying Behavioral Trends were to be developed, it could be integrated by updating the Future Model 717 for T-Log data post processing 719. The gateway would then direct T-Logs 702 identified as relevant to behavior into a new, dedicated processing pipeline, similar to how it currently handles GERs 704 and Vital Signs 720.

FIG. 7A illustrates the QAA Process Flow (Triggered from GER Side). When a New GER 712 is created for an Individual, the Therap application retrieves the GER details, along with any Existing Alerted T-Logs 721 for that Individual that are still in ‘Open’ status and share the same GER type labels. This information is fetched from the Database 709A by the application and sent to the QAA text similarity endpoint 709B to determine whether the GER describes the same events as those in the T-Logs. The endpoint returns a context similarity score, which is then evaluated against a threshold value, ‘T4’, to classify whether the GER and an alerted T-Log are a match. If a Match 727 is identified, the matched GER 722 information is stored alongside the corresponding T-Log alert in the QAA Data Store 713, and an updated notification containing the matched GER info 723 is pushed to the QA Assistant Notification Panel 724. If no match is found 726, no further action is taken 725, and the existing alerts remain unchanged.

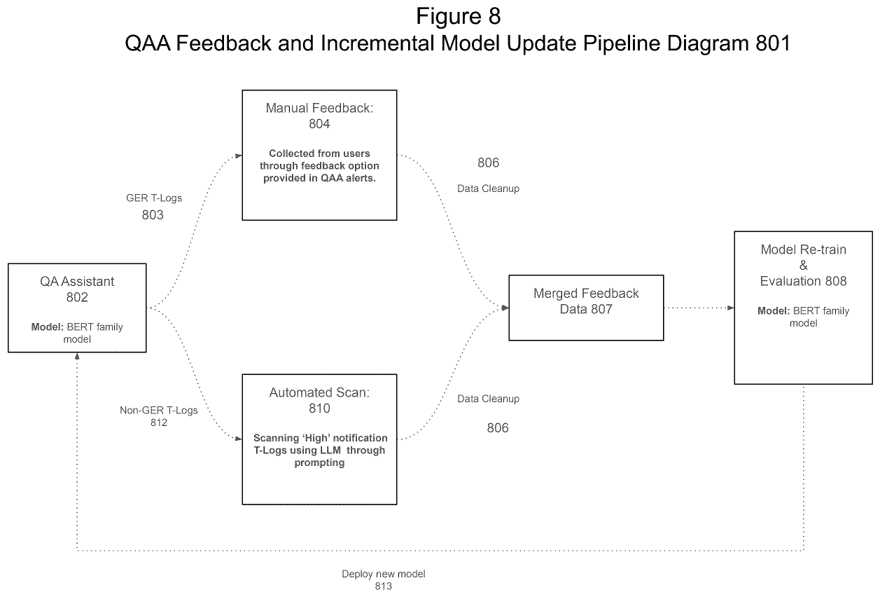

FIG. 8 illustrates the QA Assistant Feedback and Incremental Model Update Pipeline Diagram 801. This diagram describes a systematic process for the continuous improvement of a machine learning model's accuracy through a feedback pipeline. The feedback mechanism is comprised of two primary workflows: Manual Feedback 804 and Automated Scans 810. When the QA Assistant Model 802 generates an alert, such as identifying a GER T-Log 803, it is presented to Manual feedback 804 process. This interface provides options for authorized users to submit feedback on the accuracy of the generated alert. The user has the ability to correct misclassifications. For instance, if the model identifies an event as a “GER” but the user determines it is not, they can mark the alert as Not a GER. Furthermore, if the user identifies the event as a different category, such as Vital Signs, they can provide this specific, corrective suggestion. This manual feedback is crucial for capturing nuanced errors that the model may make. These feedback inputs submitted manually through the application are collected as feedback data. This data undergoes a Data Cleanup 806 process and send to Merged Feedback Data 807. To address the possibility of the primary model failing to generate alerts for actual events a second process, the Automated Scan 810, is implemented. This process is designed to identify events that the primary model may have missed. A designated sample of data points that were initially classified by the primary model as GER, non-GER, or Vital Signs is subjected to a daily Automated Scan 810. The purpose of this scan is to re-evaluate the initial classifications and determine if the primary model erroneously missed any true GER events within the sample. The data identified through this automated verification process is also collected and subjected to a similar Data Cleanup 806 procedure. The feedback data gathered from both the Manual Feedback 804 and the Automated Scan 810 workflows are consolidated and stored in a central data store referred to as Merged Feedback Data 807. This aggregated and cleaned dataset represents a valuable collection of corrected classifications and newly identified events. It is subsequently used to Model Re-train & Evaluation 808. This cyclical process—deploying new model 813, collecting feedback, retraining with corrected data, and re-evaluating—establishes a continuous improvement loop. By systematically incorporating both manual and automated feedback, the accuracy of the machine learning model is expected to increase progressively over time, leading to a more robust and reliable system.

In the system different staff members might have different level of access to information. As they can have role-based or caseload-based access for security and privacy. If a staff member creates a T-Log but not allowed to view other incident reports, especially those concerning allegations of abuse or neglect. In this scenario different staff members might hold different information. And the primary objective is to connect this information to form a complete picture without violating the access privilege, ensuring efficiency while maintaining compliance with HIPAA-type regulations. When a staff member creates a T-log without knowing that a Supervisor has already submitted an Incident Report about that same injury, classifying it as a potential case of abuse. The staff member might later decide to file their own incident report, leading to wasted time and create duplicate T-Logs. And this is where the QA Assistant fundamentally changes the workflow. The QA Assistant, which can see the data, recognizes that the T-log and the Incident Report describes the same event. It then sends a notification to the T-log submitter which can indicate that the Incident Report has already been created previously for the same event. At the same time, the system does not allow that particular staff member to click on or view the reports of that Incident Report because of the permission. So system acknowledges a related document exists to prevent redundant work. The staff member knows a report is there, but they cannot know why it's restricted. If the supervisor or case manager who does have the permission to view both T-logs and Incident Reports they can open the Incident Report about the injury, the QA Assistant will generate an alert which may include information related to the newly submitted T-Log for the same incident and if the Supervisor wants to link T-Log with the report or not.

This high-privilege user can then view the T-log, confirm its relevance, and formally link the two documents within the system. This action is vital because it ensures that all related documentation is connected, providing a comprehensive and accurate record of the event for future reviews, investigations, or audits. The system facilitates the creation of a complete record by empowering the user who has the authority to see the full picture.

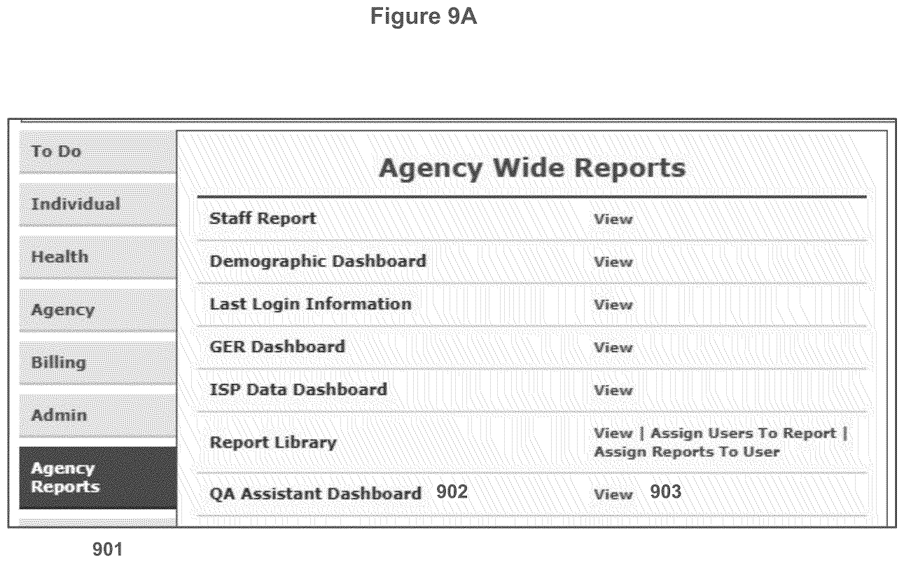

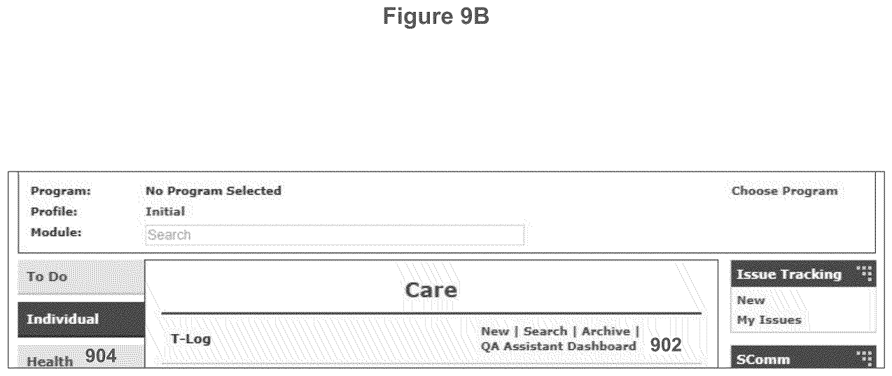

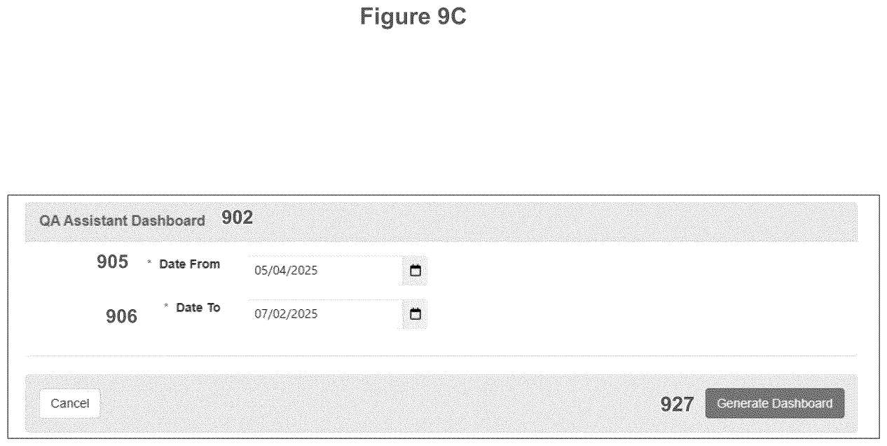

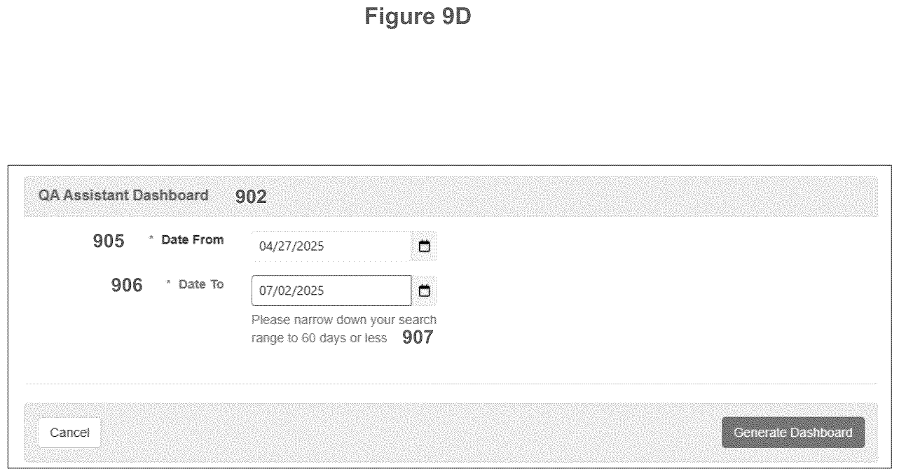

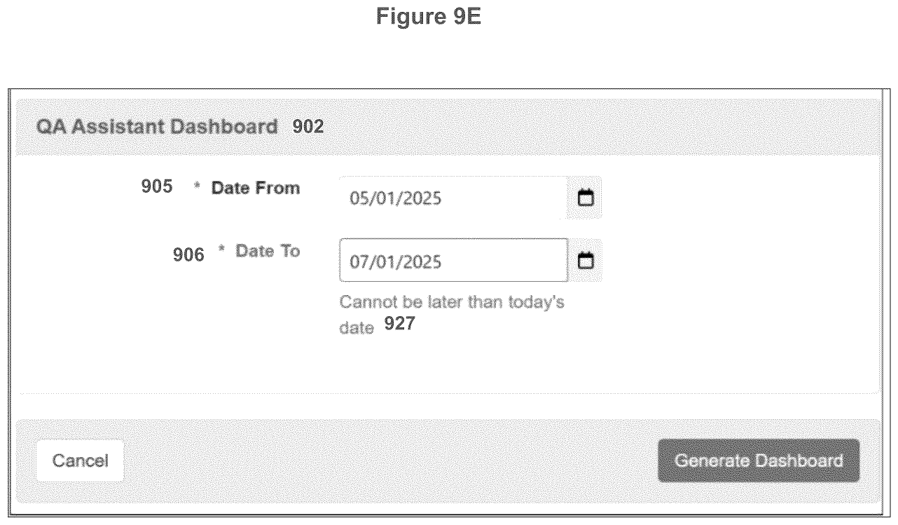

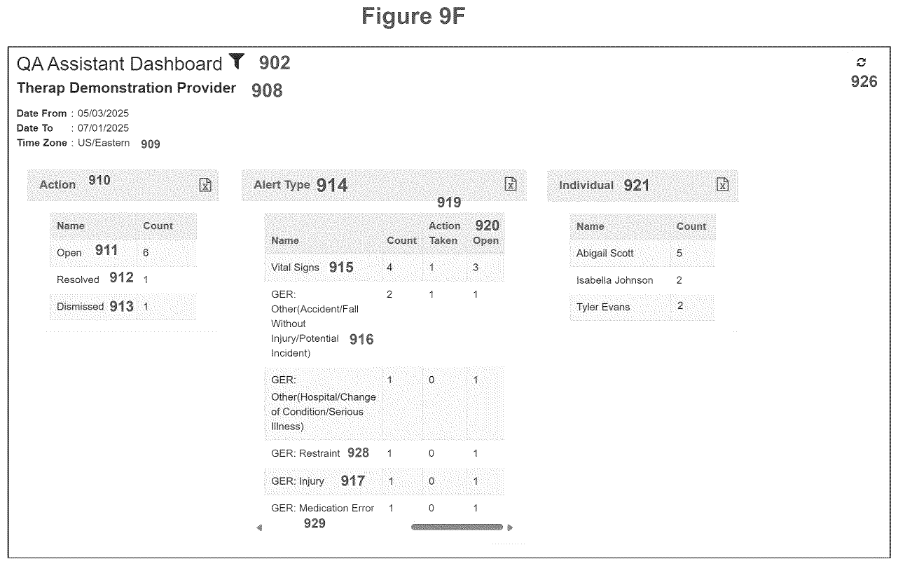

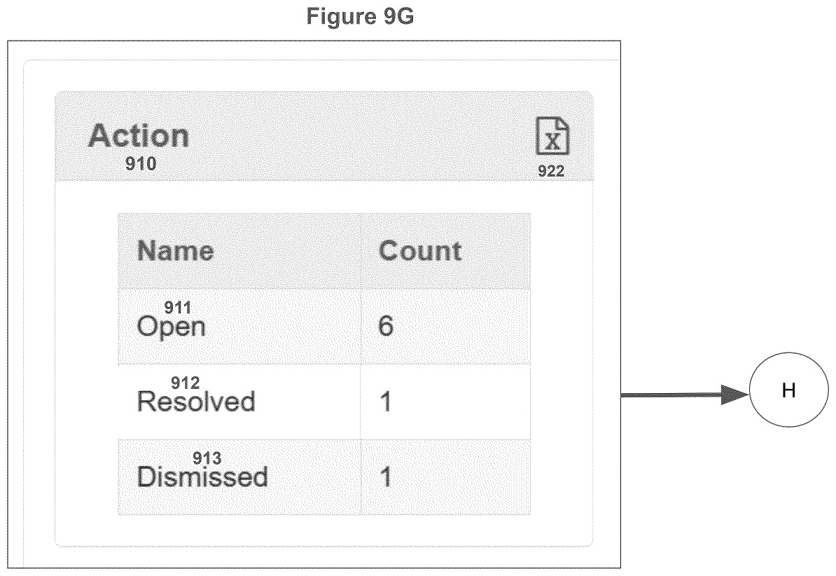

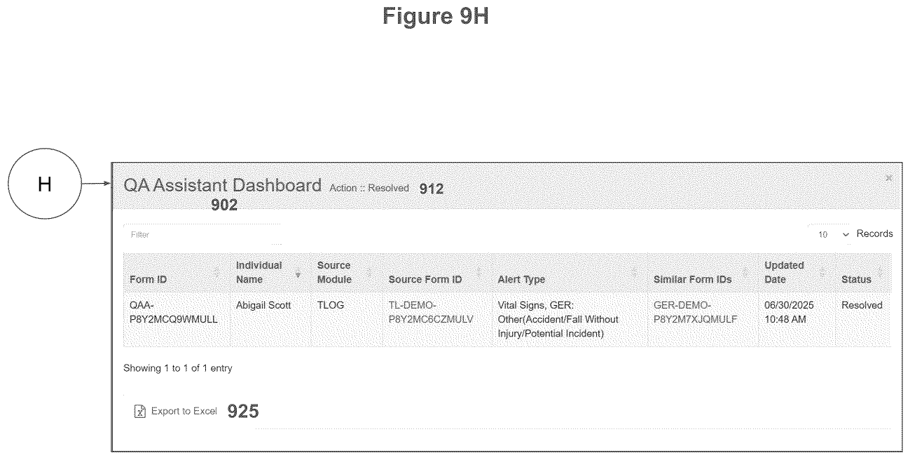

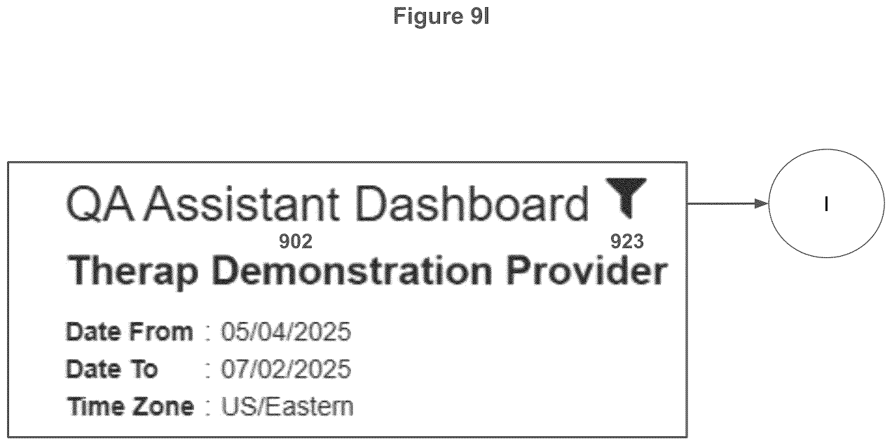

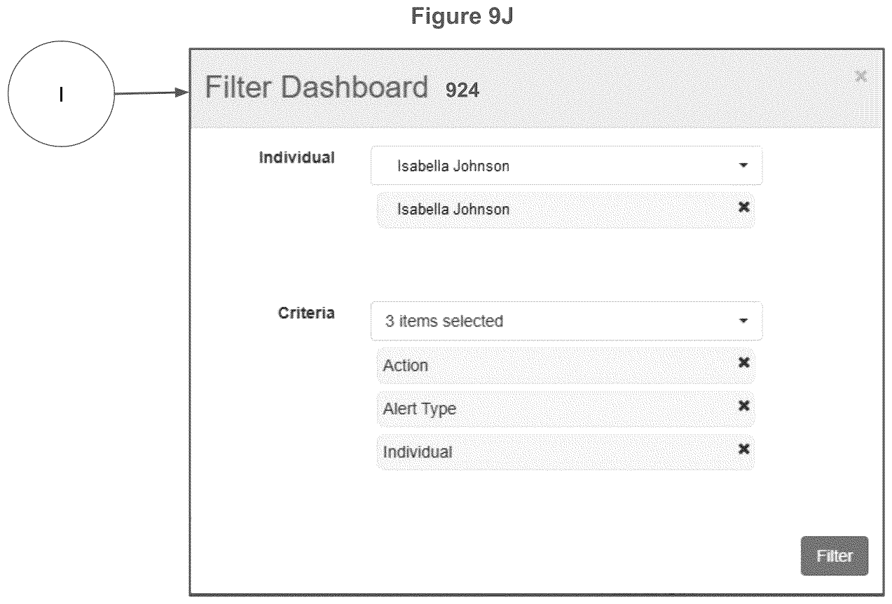

Generating QA Assistant Dashboard: FIGS. 9A-9J show exemplary screens for generating an alternative embodiment QA Assistant Dashboard.

FIG. 9A illustrates clicking on the View 903 link next to the QA Assistant Dashboard 902 option on an authorized user's Agency Reports tab 901 to open the QA Assistant Dashboard 902.

FIG. 9B illustrates QA Assistant Dashboard 902 can also be generated from an authorized user's Individual 904 tab of the main dashboard.

FIG. 9C illustrates the ‘QA Assistant Dashboard’ 902 search criteria page. The last 60 days date range is selected in the Date from 905 to Date To 906 fields, by default. Users can also manually select a different date range within 60 days and then search data using the Generate Dashboard button 927.

FIG. 9D illustrates if a user searches data for a date range that is beyond 60 days the user receives a validation 907 stating Please narrow down your search range to 60 days or less. In such cases, the user selects a valid date range, and then re-generate the dashboard.

FIG. 9E illustrates that when the user search for a date that is later than the current date, the user will receive a validation 927 stating Cannot be later than today's date.

FIG. 9F illustrates the generated QA Assistant Dashboard 902 that displays the name of the provider 908, the search parameters for which the dashboard was generated, and the time zone 909 selected in the ‘Personal Details’ page of the user who generated the Dashboard. The following criteria sections are available:
Action 910: Based on the actions taken to T-Logs using QA Assistant, the following counts are displayed:
Dismissed 913: Lists the T-Logs (See FIG. 6A and description above) for which ‘Not a GER’ 1204 or ‘Not a Vital Signs’ 1215 was selected from the QA Assistant 1201 floating panel.
Open 911: Lists the T-Logs that are available in the ‘Open’ 1202 tab of the QA Assistant Panel 1209.
Resolved 912: Lists the T-Logs that are available in the ‘Resolved’ 1203 tab of the QA Assistant Panel 1201.
Alert Type 914: Based on the detected events, counts for the following forms will be displayed:
GER: Injury 917
GER: Other 916
GER: Restraint 928
GER Medication Error 929
Vital Signs 915

Breakdown of the total T-Log counts are displayed under the Action Taken 919 and Open 920 columns. If an action has been taken to the T-Log they are added under the Actions Taken 919 column, and the T-Logs that have not been resolved 912, are added under the Open 920 column.

Individual 921: Lists the individuals whose T-Logs have been considered for potential GERs (see FIG. 5A and description above) and/or Vital Signs.

FIG. 9G illustrates that a user can open the specific list and Excel report by clicking on the Excel icon 922 in the top right corner of each section. Users can click on the Resolved 912 (indicated by H) option to open the QA Assistant Dashboard 902 for Resolved 912 status.

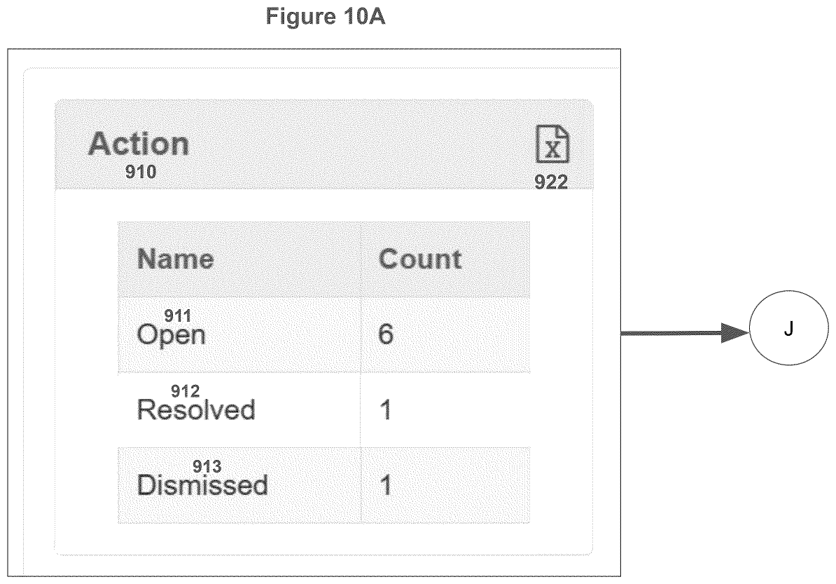

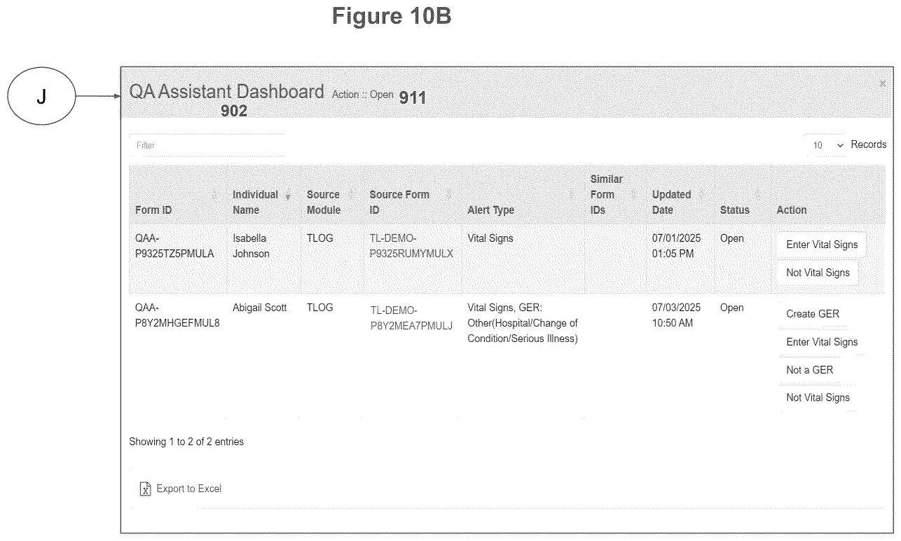

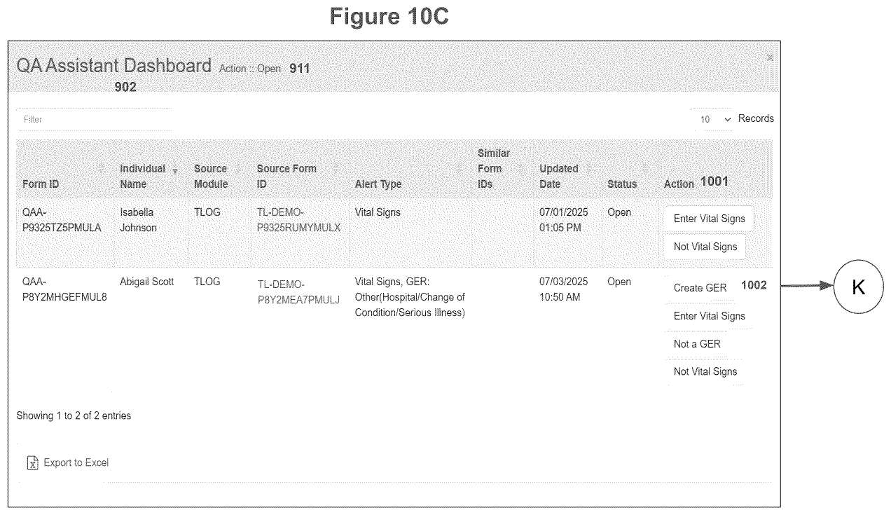

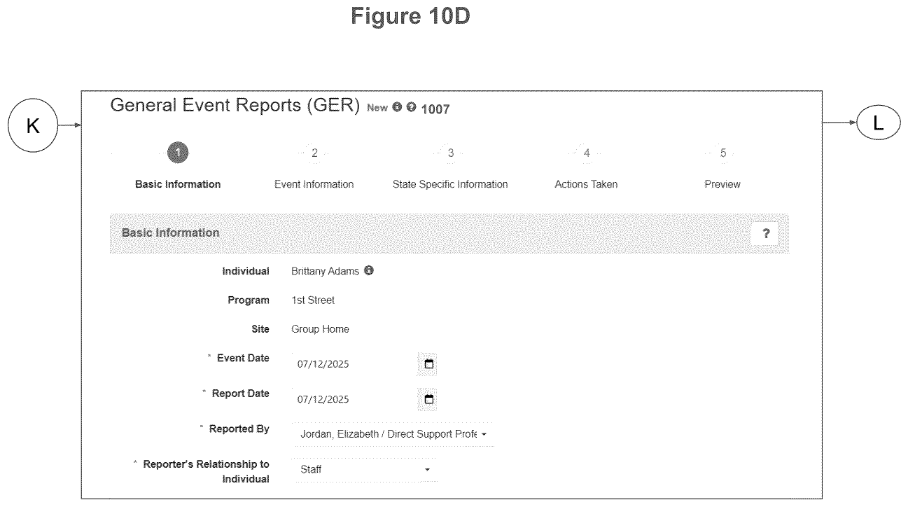

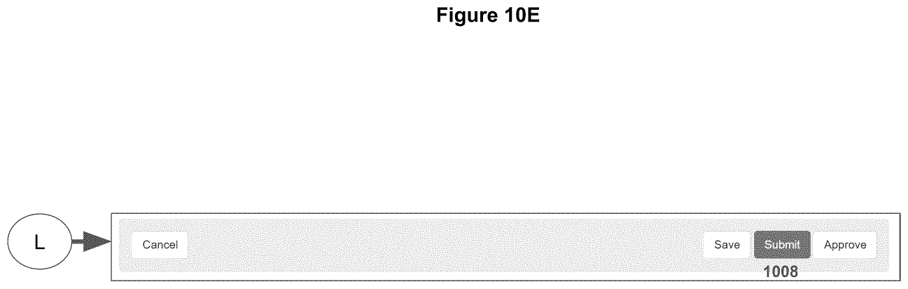

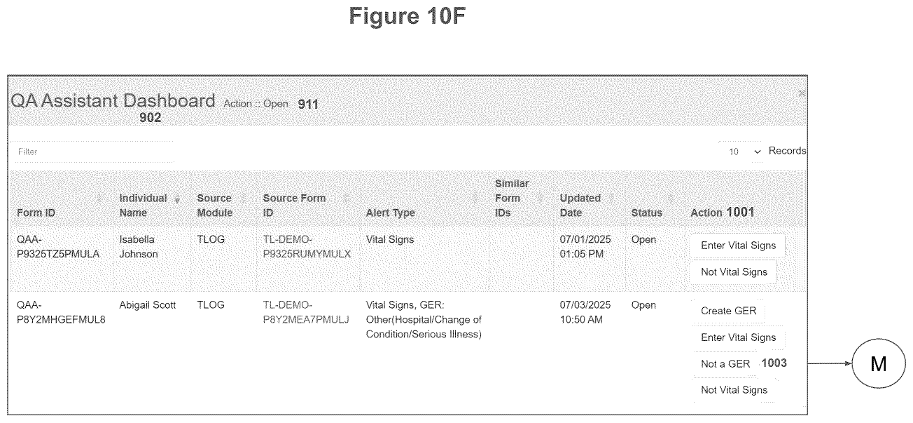

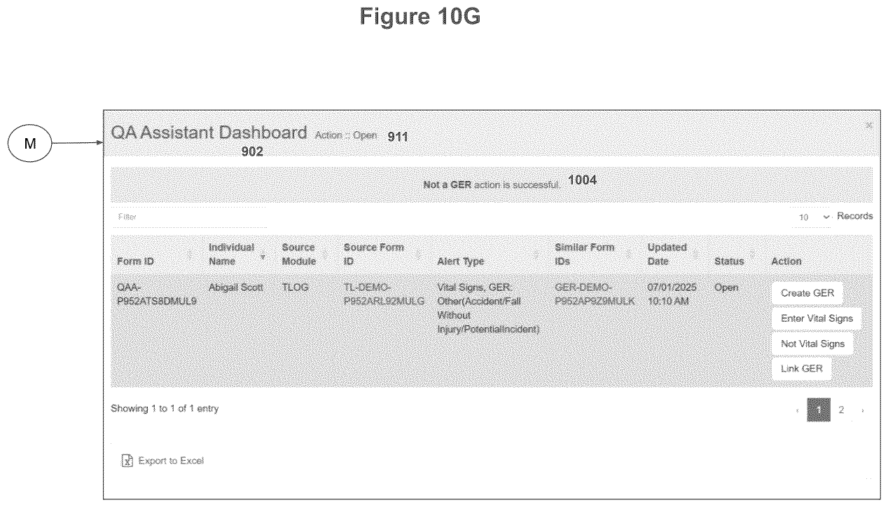

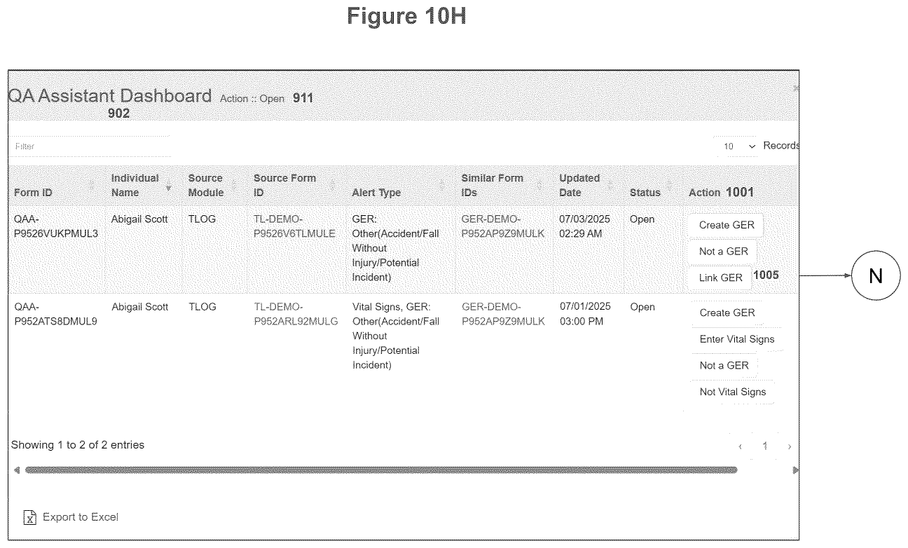

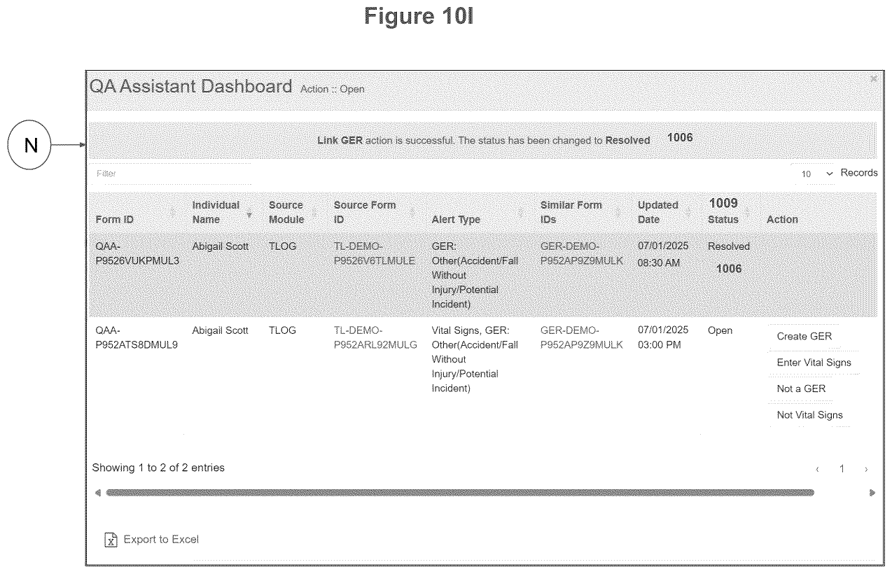

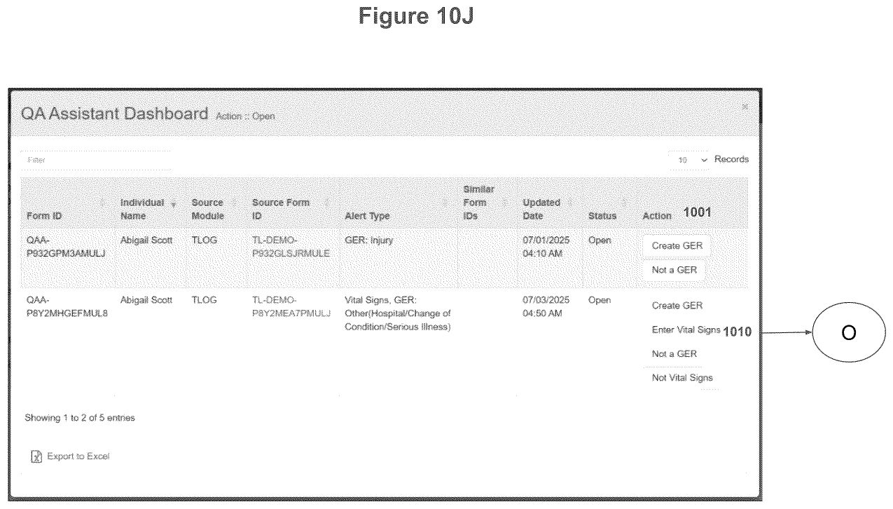

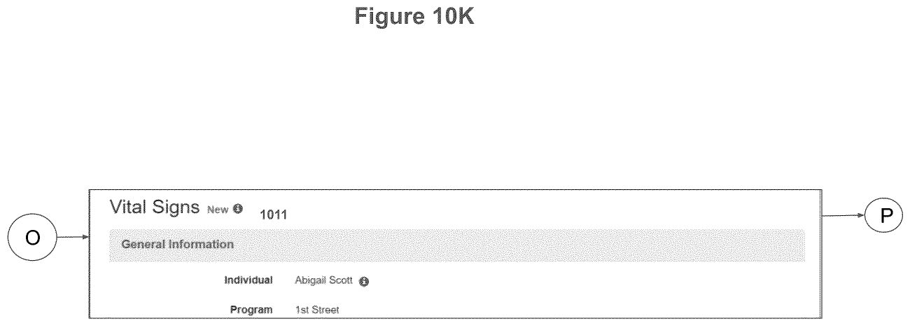

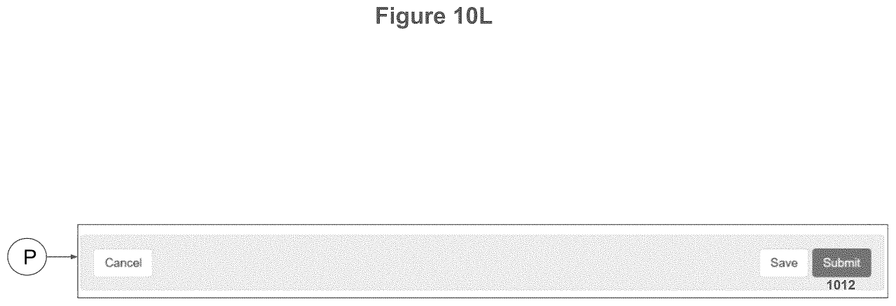

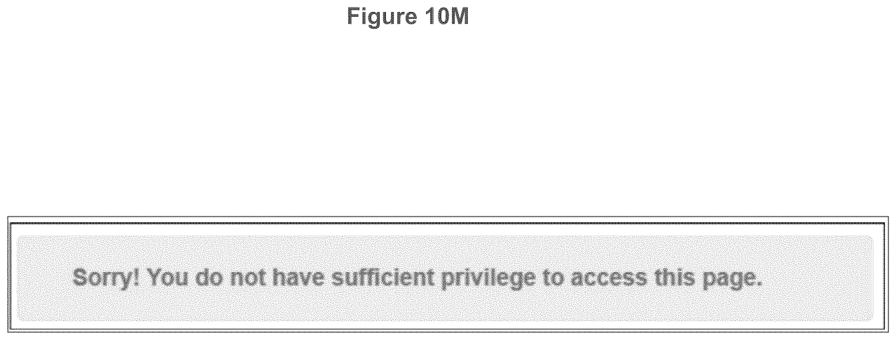

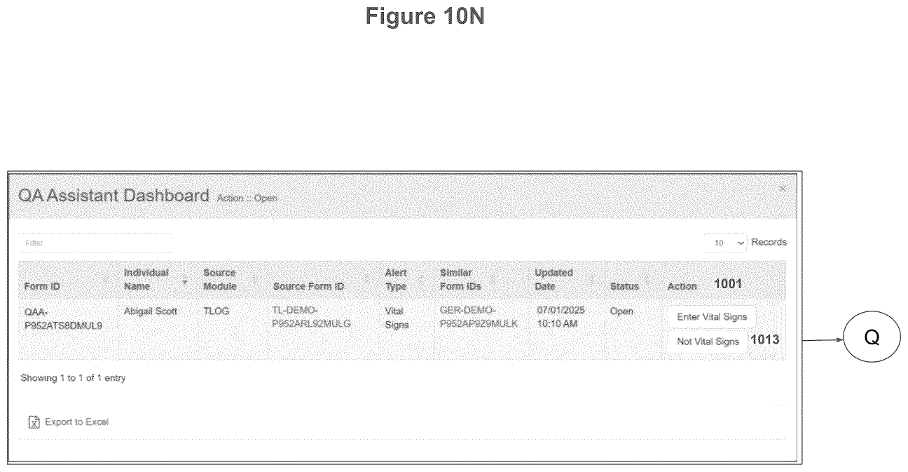

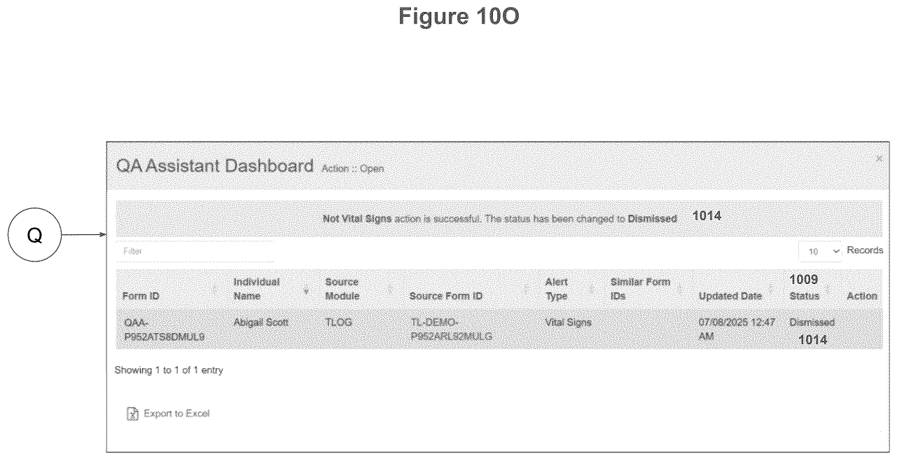

FIG. 9H shows the QA Assistant Dashboard 902 as described with reference to FIG. 10F and illustrates that clicking on the rows (indicated by H) opens up the QA Assistant Dashboard 902 in resolved status 912. Users can also export the list into excel using the Export to Excel 925 link at the bottom of the popup window. Users with the appropriate caseload-based roles (as described in the '370 Patent as referenced above) are able to click on the T-Log or GER Form ID links to open the respective forms in a new tab.

FIG. 9I illustrates that to narrow down the displaying QA Assistant Dashboard 902, users can click on the Filter icon 923 (indicated by I) on the top of the page.

FIG. 9J shows selecting the appropriate information on the ‘Filter Dashboard’ 924 pop-up window (indicated by I), and filters the QA Assistant Dashboard 902.

Users can also use the Refresh button 926 on the top right corner of the QA Assistant Dashboard 902 FIG. 3D to reset the searched data.

Create and Link GERs from QA Assistant Dashboard: FIGS. 10A-10O shows exemplary screen for creating and linking one or more GERs from the QA Assistant Dashboard 902.

FIG. 10B illustrates a ‘QA Assistant Dashboard’ 902 (indicated by J) accessed from the ‘Open’ 911 (indicated by J) row of the Action 910 criteria FIG. 10A. If any matching GER is detected, it displays under the Similar Form IDs column.

FIG. 10C illustrates that to create a GER for the recorded T-Log, the user may click on the Create GER 1002 (indicated by K) button under the Actions 1001 column. The user is then redirected to a new tab with a New GER form 1007 FIG. 10D in it to create a GER (indicated by K) and then the user can Submit 1008 (indicated by L) the GER, as shown in FIG. 10E.

FIG. 10F shows that to confirm the recorded T-Log is not a GER, the user needs to click on the Not a GER 1003 button (indicated by M) under the Action 1001 column. A success message is displayed on top of the pop-up window (indicated by M), stating that Not a GER action is successful 1004 as shown in FIG. 10G.

Users with appropriate caseload-based roles (as described in the '370 Patent as referenced above) are able to click on the T-Log link in the success message to open it in a new tab. Consequently, the count in the ‘Open’ 911 row decreases, while the count in the ‘Dismissed’ 913 row under the ‘Action’910 section increases accordingly.

FIG. 10H shows that to link a T-Log with the matching GER, the user clicks on the Link GER button 1005 (indicated by N). A success message is displayed on top of the pop-up window, stating that the status has been changed to ‘Resolved’ 1006 (indicated by N) as shown in FIG. 10I. Also the status changes to Resolved 1006 under the Status 1009 column.

Users with appropriate caseload-based roles (as described in the '370 Patent as referenced above) are able to click on the T-Log link in the success message to open it in a new tab. Consequently, the count in the ‘Open’ 911 row is decreased, while the count in the ‘Resolved’ 912 row under the ‘Action’ 910 section is increased accordingly.

FIG. 10J illustrates users can create a Vital Signs from the detected T-Log by clicking on the Enter Vital Signs button 1010 (indicated by O) under the Action 1001 column. This will open up a new Vital Signs form 1011 (indicated by O) where individual and program information will be pre-selected.

FIG. 10K illustrates creating the Vital Signs form 1011 after entering the necessary information and clicking on the Submit 1012 (indicated by P) button.

FIG. 10M illustrates if a user does not have the appropriate role assigned, an error message will be displayed stating ‘Sorry! You do not have sufficient privilege to access this page.’

FIG. 10N illustrates that a user can click on the Not Vital Signs 1013 button (indicated by Q) under the Action 1001 column if the T-Log does not concern any Vital Signs.

FIG. 10O illustrates a success message that will be displayed if the action was performed successfully. If the T-Log does not concern Vital Signs, the status 1009 of the QAA form will change from ‘Open’ to ‘Dismissed’ 1014 (indicated by Q). However, if the T-Log does involve Vital Signs, the status will remain ‘Open’ to allow for any necessary follow-up actions. The associated T-Log row will be highlighted in green to reflect the update.

Users assigned with the QA Assistant caseload-based role can access a QA Assistant floating panel throughout the Therap Services web application. Based on the submitted T-Logs, Notifications for potential GER and Vital Signs related T-Logs are displayed, and users can view the count of detected T-Logs directly from the QA Assistant floating panel.

If Notifications for Therap Services is allowed in user's web browser, then the user will be able to receive notifications after submitting or updating a T-Log that is identified as a potential GER or Vital Signs. For the T-Logs that were submitted or updated by others, users will not receive any notification.

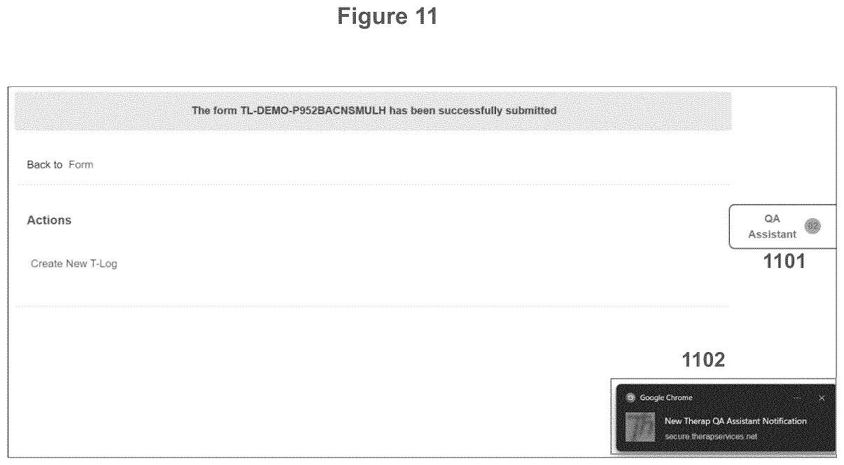

FIG. 11 illustrates when the QA Assistant detects 1101 a T-Log, a notification popup 1102 is sent to the user, and the QA Assistant count increases. Upon receiving a new notification, the count color changes to yellow. Refreshing or navigating away from the web page changes/resets the count color to blue.

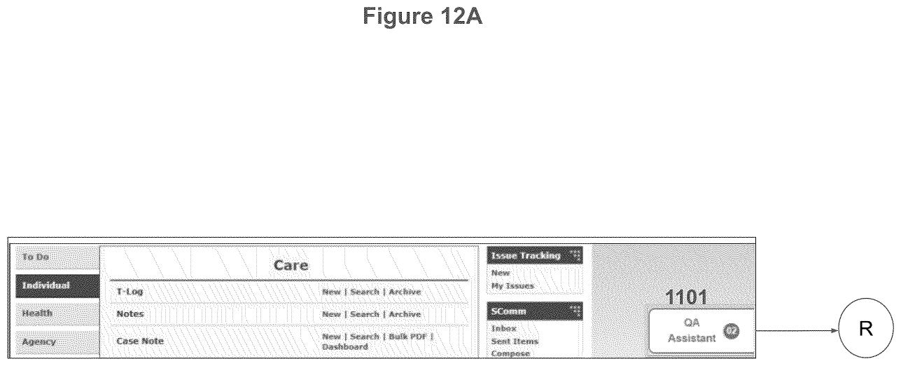

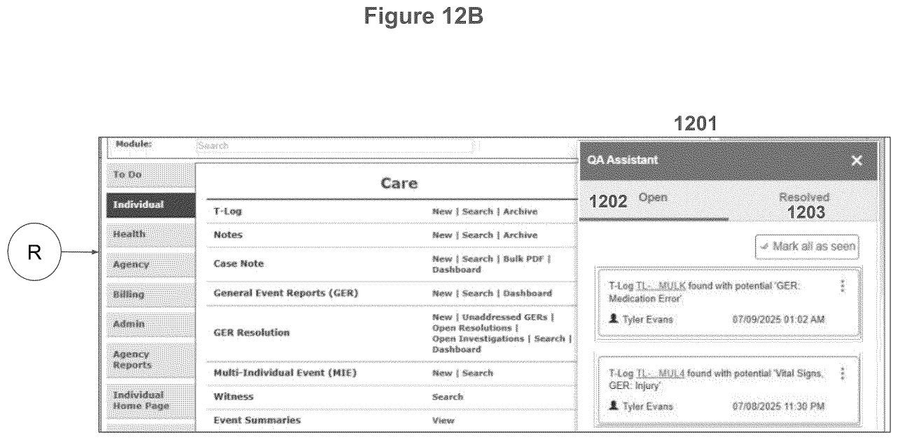

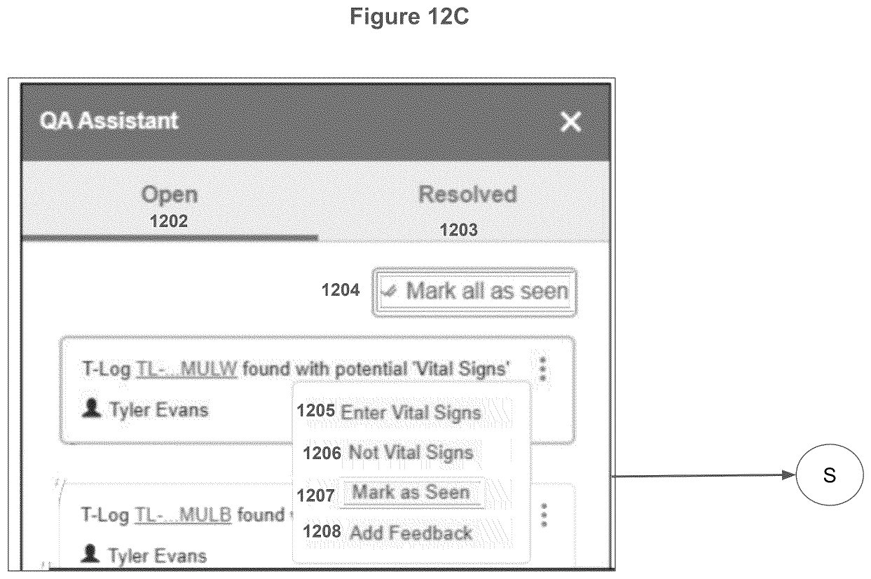

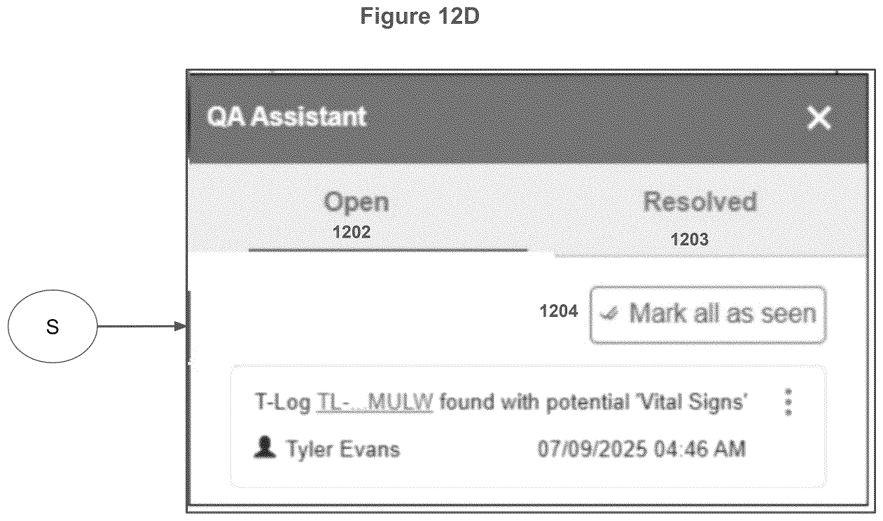

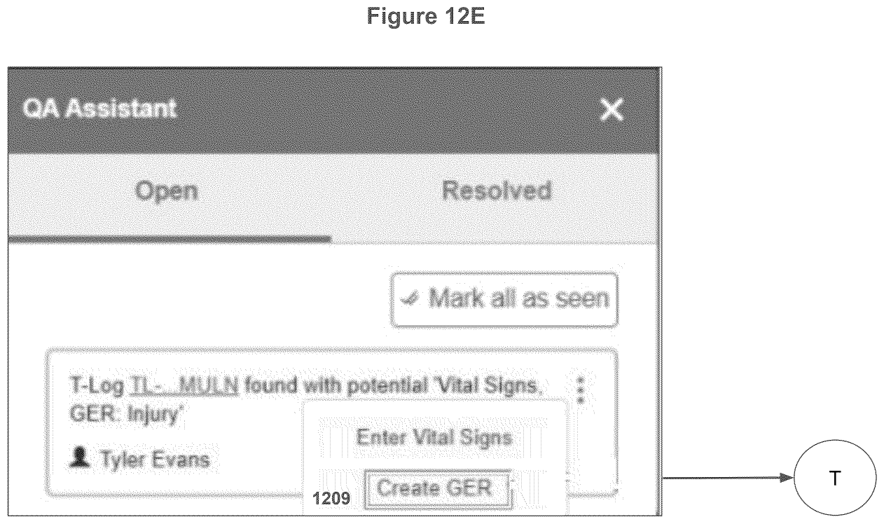

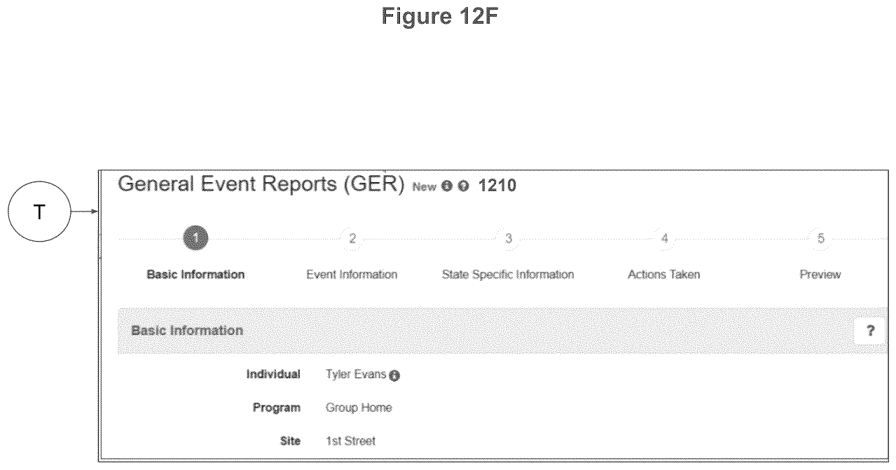

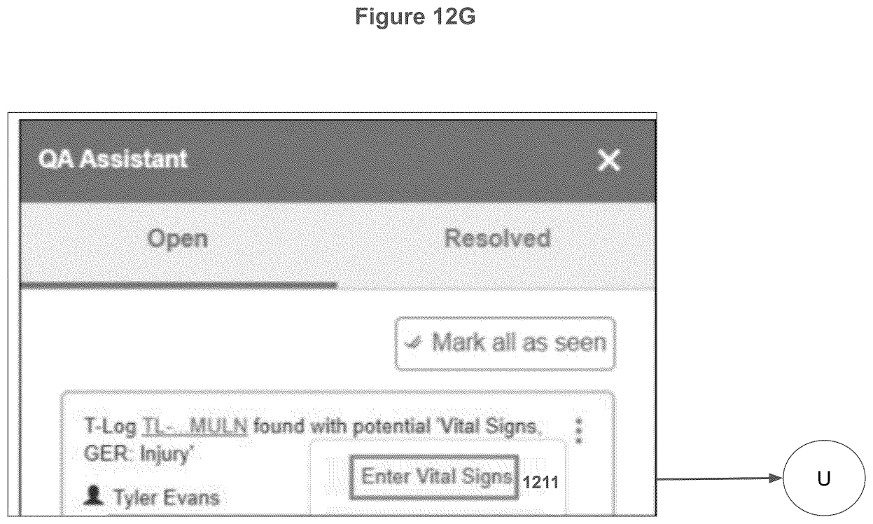

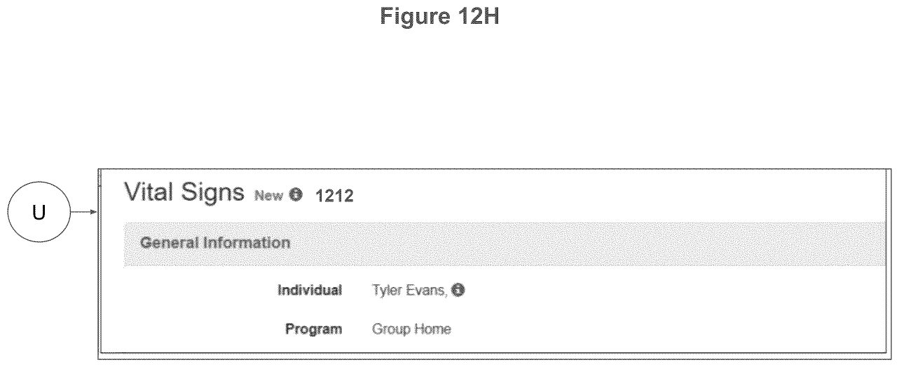

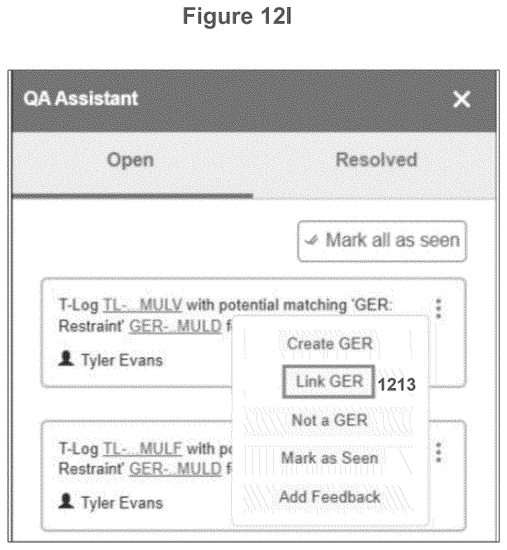

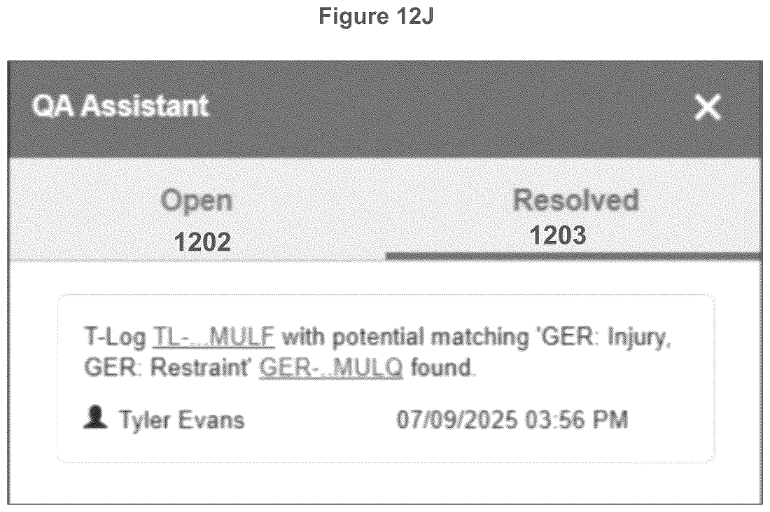

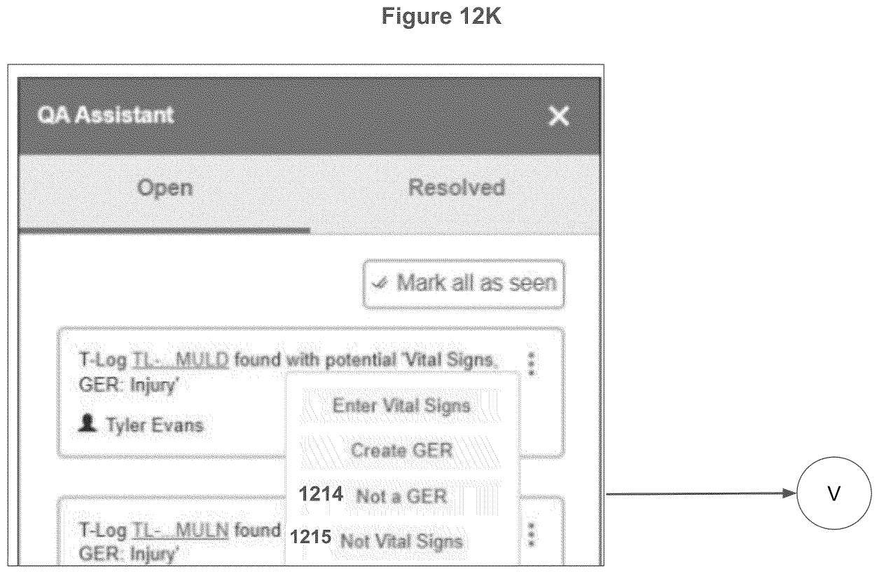

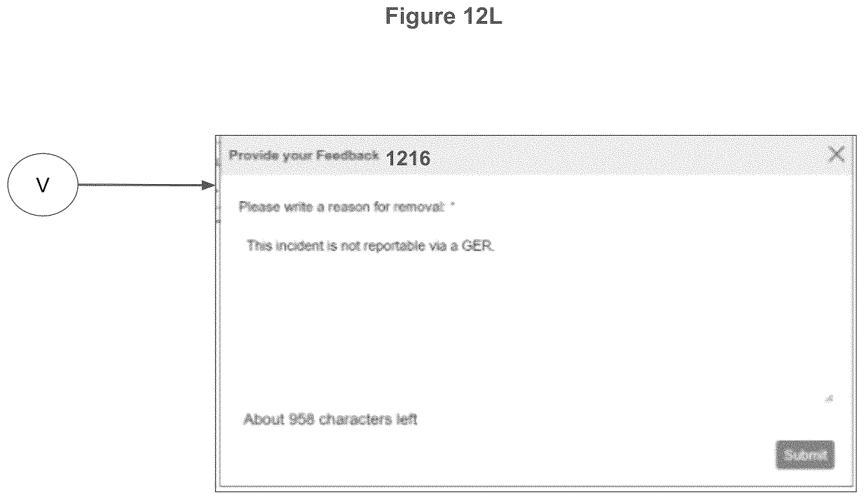

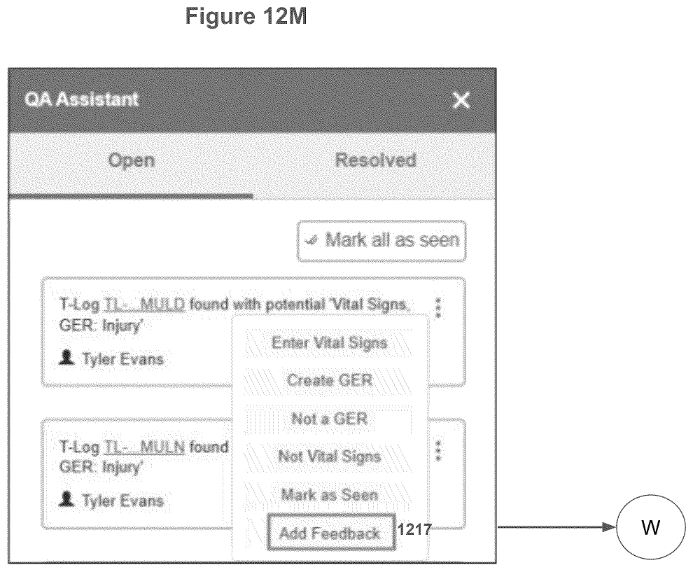

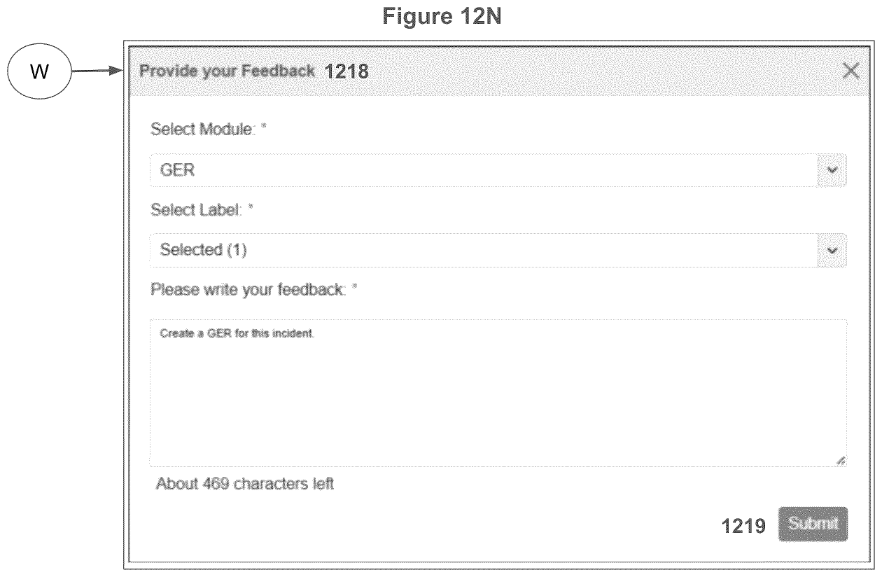

FIG. 12A-12N show exemplary screens for QA Assistant side panel features from the dashboard.

FIG. 12A illustrates that by clicking on the QA Assistant panel 1101 (indicated by R) will open up the ‘QA Assistant’ floating sidebar 1201.

FIG. 12B illustrates that the QA Assistant panel 1201 contains 2 tabs: Open 1202 and Resolved 1203. The received notifications will appear in the Open 1202 tab, and the notifications for which T-Logs have been linked to GERs by clicking on the Link GER button from the 3-dot menu, will appear in the Resolved 1203 tab.

FIG. 12C illustrate When a T-Log is identified as a potential GER, then the 3-dot menu displays the Create GER, Not a GER, Mark As Seen, and Add Feedback buttons if the users have appropriate GER roles. Similarly, if the T-Log is identified as a potential Vital Signs, the 3-dot menu displays Enter Vital Signs 1205, Not Vital Signs 1206, Mark As Seen 1207, and Add Feedback 1208 button if the users have appropriate GER roles. When a T-Log is considered for both GER and Vital Sign, all these links will show up, and if a GER exists matching the T-Log, then an additional Link GER option is also displayed. The notification will remain highlighted until the user clicks the Mark As Seen 1207 button (indicated by S) inside the 3-dot menu for the respective notification or selects the Mark All As Seen 1204 button at the top.

FIG. 12D illustrates clicking the Mark All As Seen 1204 button will remove the highlight from all notifications in the Open tab 1202.

FIG. 12E illustrates by clicking on the Create GER 1209 button (indicated by T), users with GER Submit caseload-based role will be able to create a GER 1210 for the associated individual, shown in FIG. 12F.

FIG. 12G illustrates by clicking on the Enter Vital Signs 1211 button (indicated by U), users with Health Tracking Submit caseload-based role will be able to create a Vital Signs form 1212 for the associated individual as shown in FIG. 12H.

FIG. 12I illustrates users can link a T-Log with GER by clicking on the Link GER 1213 button from the 3-dot menu of the T-Log.

FIG. 12J illustrates by clicking the Link GER 1213 button will remove the corresponding T-Log from the Open 1202 tab and update the count on the QA Assistant floating panel. User needs to navigate to the Resolved 1203 tab to locate the T-Log they have just linked to a GER. Users will not be able to link GERs for which they do not have privileges.

FIG. 12K illustrates that users can mark a T-log identified as a potential GER or Vital Signs as Not a GER or Not a Vital Signs, by clicking on the Not a GER 1214 or Not Vital Signs 1215 button (indicated by V) respectively from the 3-dot menu. And this will open a feedback box 1216 where the user needs to write the reason to remove the GER or Vital Signs as shown in FIG. 12L.

FIG. 12M illustrates users can add feedback for each T-log that is identified as a potential GER or Vital Signs by clicking on the Add Feedback 1217 (indicated by W) button from the 3-dot menu.

FIG. 12N illustrates clicking the Add Feedback 1217 button will open a Provide your Feedback popup window 1218. After entering necessary information, click on the Submit 1219 button to submit the feedback. Once feedback is submitted, the Add Feedback 1217 button will be removed from the 3-dot menu.

It will be understood by those of ordinary skill in the art that various changes may be made and equivalents may be substituted for elements without departing from the scope of the invention. In addition, many modifications may be made to adapt a particular feature or material to the teachings of the invention without departing from the scope thereof. Therefore, it is intended that the invention not be limited to the particular embodiments disclosed, but that the invention will include all embodiments falling within the scope of the claims.

1. An improvement to the way that computer systems operate to link electronic GER records with electronic T-Log records relating to individuals under care by a caregiver, the improvement comprising a HIPAA-compliant method of receiving and recording personal health information data in both GER and T-Log electronic formats relating to at least one individual, comparing the T-Log records to the GER records to determine a probable match, comparing any probable match to stored GER record types to determine a match, and linking the T-Log record to the matched GER record having a matched GER record type, the method comprising:
performing, by the computer system, the steps of:
a. providing a database of stored GER subject matters;
b. providing a database of stored GER events;
c. providing a database of stored GER subtypes;
d. providing a database of stored existing GER records;
e. providing a device for use by the caregiver;
f. providing a link database for storing links between GER records and T-Log records;
g. storing a GER subject matter threshold;
h. storing a GER event threshold;
i. storing a GER subtype threshold;
j. storing at least one GER record relating to the individual;
k. storing at least one T-Log record relating to the individual;
l. comparing said T-Log record to said GER subject matters to determine a match, including measuring a GER subject matter score relating to an accuracy of said match; and
m. determining whether said GER subject matter score exceeds said stored GER subject matter threshold;
n. comparing, if said subject matter score exceeds said stored GER subject matter threshold, said T-Log record to said GER events to determine a match, including measuring a GER event score relating to an accuracy of said match;
o. determining whether said GER event score exceeds said stored GER event threshold;
p. comparing, if said GER event score exceeds said stored GER event threshold, said T-Log record to said GER subtypes to determine a match, including measuring a GER subtype score relating to an accuracy of said match;
q. determining whether said GER subtype score exceeds said stored GER subtype threshold;
r. comparing, if said GER subtype score exceeds said stored GER subtype score threshold, said T-Log record to said existing GER records to determine a match, including measuring an existing GER score relating to an accuracy of said match;
s. determining whether said existing GER score exceeds said stored existing GER threshold;
t. storing, if said existing GER score exceeds said stored existing GER threshold, an electronic link from said T-Log record to said GER record in said link database; and
u. providing a notification of said link to said device.

2. The method of claim 1 further including performing, by the computer system, the steps of:
a. providing a database storing at least one authorization profile associated with the caregiver, wherein the caregiver is associated with one or more roles and one or more caseloads and said caseloads include access privilege information for the individual, wherein said access privilege information included in said caseload includes the identities of individuals to which the caregiver has access;
b. comparing the identity of the individual to the caregiver's authorization profile information, including comparing said identity of the individual to the caregiver's caseload; and
c. providing said notification of said link to the caregiver if said identity of the individual is stored in the caregiver's caseload.

3. The method of claim 1 wherein one or more of said steps of said step of comparing said T-Log record to said GER subject matters, comparing said T-Log record to said GER events, comparing said T-Log record to said GER subtypes, and comparing said T-Log record to said existing GER records, is performed at least in part by categorizing said T-Log record by using one or more large language models.

4. The method of claim 1 further including performing, by the computer system, the steps of:
a. providing a database of stored vital sign contents;
b. storing a vital sign content threshold;
c. comparing said T-Log record to said vital sign contents to determine a match, including measuring a vital sign content score relating to an accuracy of said match; and
d. determining whether said vital sign content score exceeds said stored vital sign content threshold;
e. providing, to said device, a notification that said T-Log contains vital sign data.

5. The method of claim 1 further including performing, by the computer system, the step of providing, if said existing GER score does not exceed said stored existing GER threshold, a notification to said device that said T-Log record contains GER subject matter.

6. The method of claim 5 further including performing, by the computer system, the step of providing, if said existing GER score does not exceed said stored existing GER threshold, a notification to said device suggesting that that a new GER record should be prepared based on said T-Log record subject matter.

7. An improvement to the way that computer systems operate to link electronic T-Log records with electronic GER records relating to individuals under care by a caregiver, the improvement comprising a HIPAA-compliant method of receiving and recording personal health information data in both T-Log and GER electronic formats relating to at least one individual, comparing the GER records to the T-Log records to determine a probable match, and linking the GER record to the matched T-Log record, the method comprising:
performing, by the computer system, the steps of:
a. providing a database of stored GER events;
b. providing a database of stored existing T-Log records having an open alert status;
c. providing a device for use by the caregiver;
d. providing a link database for storing links between T-Log records and GER records;
e. storing a GER event threshold;
f. storing at least one T-Log record relating to the individual having an open alert status;
g. storing at least one GER record relating to the individual;
h. comparing said GER record to said at least one T-Log record having an open alert status to determine a match, including measuring a GER event score relating to an accuracy of said match;
i. determining whether said GER event score exceeds said stored GER event threshold;
j. storing, if said GER event score exceeds said stored GER event threshold, an electronic link from said T-Log record to said GER record in said link database; and
k. providing a notification of said link to said device.

8. The method of claim 7 further including performing, by the computer system, the steps of:
a. providing a database storing at least one authorization profile associated with the caregiver, wherein the caregiver is associated with one or more roles and one or more caseloads and said caseloads include access privilege information for the individual, wherein said access privilege information included in said caseload includes the identities of individuals to which the caregiver has access;
b. comparing the identity of the individual to the caregiver's authorization profile information, including comparing said identity of the individual to the caregiver's caseload; and
c. providing said notification of said link to the caregiver if said identity of the individual is stored in the caregiver's caseload.

9. The method of claim 7 wherein said step of comparing said GER record to said existing T-Log records is performed at least in part by categorizing said GER record by using one or more large language models.

10. The method of claim 7 further including performing, by the computer system, the step of leaving said open alert status unchanged, if said existing GER event score does not exceeds said stored GER event threshold.

11. An improvement to computer systems for linking electronic GER records with electronic T-Log records relating to individuals under care by a caregiver, the improvement comprising a HIPAA-compliant computer program adapted to receive and record personal health information data in both GER and T-Log electronic formats relating to at least one individual, compare the T-Log records to the GER records to determine a probable match, compare any probable match to stored GER record types to determine a match, and link the T-Log record to the matched GER record having a matched GER record type, the system comprising:
a. a computer system having a memory and a processor;
b. a database of stored GER subject matters;
c. a database of stored GER events;
d. a database of stored GER subtypes;
e. a device for use by the caregiver;
f. a link database for storing links between GER records and T-Log records;
g. a database for storing a GER subject matter threshold;
h. a database for storing a GER event threshold;
i. a database for storing a GER subtype threshold;
j. a database for storing at least one GER record relating to the individual;
k. a database for storing at least one T-Log record relating to the individual;
l. a computer program configured to run on said computer system and configured to:
i. compare said T-Log record to said GER subject matters to determine a match, including measuring a GER subject matter score relating to an accuracy of said match; and
ii. determine whether said GER subject matter score exceeds said stored GER subject matter threshold;
iii. compare, if said subject matter score exceeds said stored GER subject matter threshold, said T-Log record to said GER events to determine a match, including measuring a GER event score relating to an accuracy of said match;
iv. determine whether said GER event score exceeds said stored GER event threshold;
v. compare, if said subject matter score exceeds said stored GER event threshold, said T-Log record to said GER subtypes to determine a match, including measuring a GER subtype score relating to an accuracy of said match;
vi. determine whether said GER subtype score exceeds said stored GER subtype threshold;
vii. compare, if said GER subtype score exceeds said stored GER subtype score threshold, said T-Log record to said existing GER records to determine a match, including measuring an existing GER score relating to an accuracy of said match;
viii. determine whether said existing GER score exceeds said stored existing GER threshold;
ix. store, if said existing GER score exceeds said stored existing GER threshold, an electronic link from said T-Log record to said GER record in said link database; and
x. provide a notification of said link to said device.

12. The system of claim 10 wherein:
a. said system further comprises a database storing at least one authorization profile associated with the caregiver, wherein the caregiver is associated with one or more roles and one or more caseloads and said caseloads include access privilege information for the individual, wherein said access privilege information included in said caseload includes the identities of individuals to which the caregiver has access; and
b. said computer program is further configured to:
i. compare the identity of the individual to the caregiver's authorization profile information, including comparing said identity of the individual to the caregiver's caseload; and
ii. provide said notification of said link to the caregiver if said identity of the individual is stored in the caregiver's caseload.

13. The system of claim 10 wherein said computer program further includes one or more large language models, and one or more of said comparing said T-Log record to said GER subject matters, said comparing said T-Log record to said GER events, said comparing said T-Log record to said GER subtypes, and said comparing said T-Log record to said existing GER records, is performed by said computer program at least in part by categorizing said T-Log record by using said one or more large language models.

14. The system of claim 13 wherein:
a. said system further comprises:
i. a database of stored vital sign contents;
ii. a database for storing a vital sign content threshold; and
b. said computer program is further configured to:
i. compare said T-Log record to said vital sign contents to determine a match, including measuring a vital sign content score relating to an accuracy of said match; and
ii. determine whether said vital sign content score exceeds said stored vital sign content threshold; and
iii. provide, to said device, a notification that said T-Log contains vital sign data.

15. The system of claim 10 wherein said computer program is further configured to provide, if said existing GER score does not exceed said stored existing GER threshold, a notification to said device that said T-Log record contains GER subject matter.

16. The system of claim 14 wherein said computer program is further configured to provide, if said existing GER score does not exceed said stored existing GER threshold, a notification to said device suggesting that that a new GER record should be prepared based on said T-Log subject matter.

17. An improvement to computer systems for linking electronic T-Log records with electronic GER records relating to individuals under care by a caregiver, the improvement comprising a HIPAA-compliant computer program adapted to receive and record personal health information data in both T-Log and GER electronic formats relating to at least one individual, compare the GER records to the T-Log records to determine a probable match, and link the GER record to the matched T-Log record having a matched T-Log record type, the system comprising:
a. a computer system having a memory and a processor;
b. a database of stored GER events;
c. a database of stored existing T-Log records having an open alert status;
d. a device for use by the caregiver;
e. a link database for storing links between T-Log records and GER records;
f. a database for storing a GER event threshold;
g. a database for storing at least one GER record relating to the individual;
h. a computer program configured to run on said computer system and configured to:
i. compare said GER record to said at least one T-Log record having an open alert status to determine a match, including measuring a GER event score relating to an accuracy of said match; and
ii. determine whether said GER event score exceeds said stored GER event threshold;
iii. store, if said GER event score exceeds said stored GER event threshold, an electronic link from said T-Log record to said GER record in said link database; and
iv. provide a notification of said link to said device.

18. The system of claim 17 wherein:
a. said system further comprises a database storing at least one authorization profile associated with the caregiver, wherein the caregiver is associated with one or more roles and one or more caseloads and said caseloads include access privilege information for the individual, wherein said access privilege information included in said caseload includes the identities of individuals to which the caregiver has access; and
b. said computer program is further configured to:
i. compare the identity of the individual to the caregiver's authorization profile information, including comparing said identity of the individual to the caregiver's caseload; and
ii. provide said notification of said link to the caregiver if said identity of the individual is stored in the caregiver's caseload.

19. The system of claim 17 wherein said computer program further includes one or more large language models, and said comparing said GER record to said existing T-Log records is performed at least in part by categorizing said GER record by using one or more large language models.

20. The system of claim 17 wherein said computer program is further configured to leave said open alert status unchanged, if said existing GER event score does not exceeds said stored GER event threshold.
